(19) United States
(12) Patent Application Publication
(10) Pub. No.: US 2021/0401710 A1
(43) Pub. Date: Dec. 30, 2021

(54) AZOXYSTROBIN EFFICACY IN PERSONAL CARE FORMS

(71) Applicant: The Procter & Gamble Company, Cincinnati, OH (US)

(72) Inventors: Eric Scott Johnson, Hamilton, OH (US); Jeanette Anthea Richards, Liberty Township, OH (US); Geoffrey Marc Wise, Reading, OH (US); Debora W. Chang, Mason, OH (US); James Patrick Henry, Mason, OH (US); Tamara Lynn Caterino, Loveland, OH (US)

(21) Appl. No.: 17/126,387

(22) Filed: Dec. 18, 2020

Related U.S. Application Data
(60) Provisional application No. 63/044,814, filed on Jun. 26, 2020.

(51) Int. Cl. A61K 8/49; A61Q 5/02; A61Q 19/10; A61Q 5/12; A61K 8/89

(52) U.S. Cl. CPC A61K 8/4953; A61Q 5/02; A61K 8/89; A61Q 5/12; A61Q 19/10

(57) ABSTRACT

The present invention is directed to a personal care composition comprising azoxystrobin wherein at least about 0.05% azoxystrobin provides a deposition of greater than about 0.01 ug/cm2.

### FIELD OF THE INVENTION

The present invention is directed to azoxystrobin delivered from personal care forms such as shampoos, conditioners, leave on treatments, dry shampoos, single unit dose and personal cleansing compositions.

### BACKGROUND OF THE INVENTION

Personal care products provide solutions to consumer's scalp and skin care needs through delivery of actives to the surface of the skin and scalp. These benefits include anti-dandruff efficacy, itch relief, moisturization or soothing of sensitive skin or scalp. A key need is to utilize potent actives to provide the strongest benefit for the consumer, but these actives often come with cosmetic trade-offs in consumer acceptance such as odor or color. Selenium sulfide is considered a very potent and efficacious active for providing scalp and skin benefits but comes with a strong sulfurous odor and bright orange color. It has been surprisingly found that Azoxystrobin has comparable potency from personal care products and even more surprising is that it has none of the cosmetic issues of odor or color.

### SUMMARY OF THE INVENTION

The present invention is directed to a personal care composition comprising azoxystrobin wherein at least about 0.05% azoxystrobin provides a deposition of greater than about 0.01 ug/cm2.

### DETAILED DESCRIPTION OF THE INVENTION

While the specification concludes with claims which particularly point out and distinctly claim the invention, it is believed the present invention will be better understood from the following description.

The present invention can comprise, consist of, or consist essentially of the essential elements and limitations of the invention described herein, as well any of the additional or optional ingredients, components, or limitations described herein.

All percentages and ratios used herein are by weight of the total composition, unless otherwise designated. All measurements are understood to be made at ambient conditions, where “ambient conditions” means conditions at about 25° C., under about one atmosphere of pressure, and at about 50% relative humidity (RH), unless otherwise designated. All numeric ranges are inclusive of narrower ranges; delineated upper and lower range limits are combinable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods.

“Apply” or “application” as used in reference to a composition, means to apply or spread the compositions of the present invention onto keratinous tissue such as the hair.

“Dermatologically acceptable” means that the compositions or components described are suitable for use in contact with human skin tissue without undue toxicity, incompatibility, instability, allergic response, and the like.

“Safe and effective amount” means an amount of a compound or composition sufficient to significantly induce a positive benefit.

“Leave-on,” in reference to compositions, means compositions intended to be applied to and allowed to remain on the keratinous tissue. These leave-on compositions are to be distinguished from compositions, which are applied to the hair and subsequently (in a few minutes or less) removed either by washing, rinsing, wiping, or the like. Leave-on compositions exclude rinse-off applications such as shampoos, rinse-off conditioners, facial cleansers, hand cleansers, body wash, or body cleansers. The leave-on compositions may be substantially free of cleansing or detersive surfactants. For example, “leave-on compositions” may be left on the keratinous tissue for at least 15 minutes. For example, leave-on compositions may comprise less than 1% detersive surfactants, less than 0.5% detersive surfactants, or 0% detersive surfactants. The compositions may, however, contain emulsifying, dispersing or other processing surfactants that are not intended to provide any significant cleansing benefits when applied topically to the hair.

“Soluble” means at least about 0.1 g of solute dissolves in 100 ml of solvent, at 25° C. and 1 atm of pressure.

All percentages are by weight of the total composition, unless stated otherwise. All ratios are weight ratios, unless specifically stated otherwise. All ranges are inclusive and combinable. The number of significant digits conveys neither a limitation on the indicated amounts nor on the accuracy of the measurements. The term “molecular weight” or “M.Wt.” as used herein refers to the weight average molecular weight unless otherwise stated. The weight average molecular weight may be measured by gel permeation chromatography. “QS” means sufficient quantity for 100%.

The term “substantially free from” or “substantially free of” as used herein means less than about 1%, or less than about 0.8%, or less than about 0.5%, or less than about 0.3%, or about 0%, by total weight of the composition.

“Hair,” as used herein, means mammalian hair including scalp hair, facial hair and body hair, particularly on hair on the human head and scalp.

“Cosmetically acceptable,” as used herein, means that the compositions, formulations or components described are suitable for use in contact with human keratinous tissue without undue toxicity, incompatibility, instability, allergic response, and the like. All compositions described herein which have the purpose of being directly applied to keratinous tissue are limited to those being cosmetically acceptable.

“Derivatives,” as used herein, includes but is not limited to, amide, ether, ester, amino, carboxyl, acetyl, acid, salt and/or alcohol derivatives of a given compound.

“Polymer,” as used herein, means a chemical formed from the polymerisation of two or more monomers. The term “polymer” as used herein shall include all materials made by the polymerisation of monomers as well as natural polymers. Polymers made from only one type of monomer are called homopolymers. Polymers made from two or more different types of monomers are called copolymers. The distribution of the different monomers can be calculated statistically or block-wise—both possibilities are suitable for the present invention. Except if stated otherwise, the term “polymer” used herein includes any type of polymer including homopolymers and copolymers.

### Product Forms

The personal care composition of the present invention may be shampoo, a rinse-off hair conditioner, a leave-on treatment, a personal cleansing composition, a bar soap or a single unit dose, grooming examples including moisturizer/balm, washing compositions pre-shave compositions and post-foaming shave gels.

### Azoxystrobin and Other Strobilurins

Azoxystrobin, CAS number: 131860-33-8, IUPAC: methyl-(E)-(2-{2[6-(2-cyanophenoxy)-pyrimidin-4-iloxy]-phenyl}-3-methoxyacrylate is an agricultural fungicide belonging to the class of the strobilurins. Strobilurins are either biosynthesized by various Basidiomycete fungi such as Strobilurus tenacellus and Oudemansiella mucida or modeled after natural strobilurins and synthesized with retention of the key β-methoxyacrylate toxophore. Some synthesized strobilurins have a modified toxophore e.g. methyl methoxyiminoacetate or methyl-N-methoxycarbamate. Some synthetic strobilurins are azoxystrobin (CAS number: 131860-33-8), coumoxystrobin (CAS number 850881-70-8), dimoxystrobin (CAS number 149961-52-4), enoxastrobin (CAS number 238410-11-2), fluoxastrobin (CAS number 193740-76-0), kresoxim methyl (CAS number 143390-89-0), mandestrobin (CAS number 173662-97-0), metominostrobin (CAS number 133408-50-1), orysastrobin (CAS number 248593-16-0)), picoxystrobin (CAS number 117428-22-5), pyraclostrobin (CAS number 175013-18-0), pyraoxystrobin (CAS number 862588-11-2), and trifloxystrobin (CAS number 141517-21-7).

Azoxystrobin and other synthetic strobilurins control a broad spectrum of plant fungal disease and are used heavily in crop protection worldwide. Strobilurins work by inhibition of mitochondrial respiration. The specific mode of action of azoxystrobin and other strobilurins is by binding the ubiquinol oxidizing site (Q₀ site) in the cytochrome b complex III of the electron transport chain and blocking electron transfer between cytochrome b and cytochrome c₁. Other compounds with this specific mode of action include synthetic and naturally occurring derivatives of the key β-methoxyacrylate toxophore known as oudemansins also first isolated from Oudemansiella mucida, synthetic and naturally occurring myxothiazols from myxobacteria such as Myxococcus flavus, stigmatellins from myxobacteria such as Stigmatella aurantica and the synthetic agricultural chemicals famoxadone and fenamidone.

Azoxystrobin as an agricultural fungicide has protectant, curative, eradicant, translaminar and systemic properties and inhibits spore germination and mycelial growth, and also shows antisporulant activity. At labelled application rates, azoxystrobin controls the numerous plant pathogens including Erysiphe graminis, Puccinia spp., Lepiosphaeria nodorum, Septoria tritici and Pyrenophora teres on temperate cereals; Pyricularia oryzae and Rhizoctonia solani on rice; Plasmopara viticola and Uncinula necator on vines; Sphaerotheca fuliginea and Pseudoperonospora cubensis on cucurbitaceae; Phytophthora infestans and Alternaria solani on potato and tomato; Mycosphaerella arachidis, Rhizoctonia solani and Sclerotium rolfsii on peanut; Monilinia spp, and Cladosporium carpophilum on peach; Pythium spp. and Rhizoctonia solani on turf; Mycosphaerella spp. on banana; Cladosporium caryigenum on pecan; Elsinoë fawcetii, Colletotrichum spp. and Guignardia citricarpa on citrus; Colletotrichum spp. and Hemileia vastatrix on coffee. Azoxystrobin is a solid material having low solubility in water.
Some tradenames for azoxystrobin include ABOUND FLOWABLE FUNGICIDE, Aframe, Azoxystar, Azoxyzone, AZteroid 1.65 SC Fungicide, AZURE AGRICULTURAL FUNGICIDE, Endow, QUADRIS FLOWABLE FUNGICIDE, Satori Fungicide, Strobe 2L, and Willowood Azoxy 2SC. Azoxystrobin is commercially available from for example Sigma-Aldrich (St. Louis, Mo.) and Ak Scientific, Inc (Union City, Calif.).

In the present invention, the personal care composition may contain from about 0.02% to about 10% of azoxystrobin; from about 0.05% to about 2% of azoxystrobin; from about 0.1% to about 1% of azoxystrobin. Further, the personal care composition may contain from about 0.02% to about 10% of a strobilurin; from about 0.05% to about 2% of a strobilurin; from about 0.1% to about 1% of a strobilurin.

### Shampoo Compositions

### Detersive Surfactant

The personal care composition may comprise greater than about 10% by weight of a surfactant system which provides cleaning performance to the composition, and may be greater than 12% by weight of a surfactant system which provides cleaning performance to the composition. The surfactant system comprises an anionic surfactant and/or a combination of anionic surfactants and/or a combination of anionic surfactants and co-surfactants selected from the group consisting of amphoteric, zwitterionic, nonionic and mixtures thereof. Various examples and descriptions of detersive surfactants are set forth in U.S. Pat. No. 8,440,605; U.S. Patent Application Publication No. 2009/155383; and U.S. Patent Application Publication No. 2009/0221463, which are incorporated herein by reference in their entirety.

The personal care composition may comprise from about 10% to about 25%, from about 10% to about 18%, from about 10% to about 14%, from about 10% to about 12%, from about 11% to about 20%, from about 12% to about 20%, and/or from about 12% to about 18% by weight of one or more surfactants.

Anionic surfactants suitable for use in the compositions are the alkyl and alkyl ether sulfates. Other suitable anionic surfactants are the water-soluble salts of organic, sulfuric acid reaction products. Still other suitable anionic surfactants are the reaction products of fatty acids esterified with isethionic acid and neutralized with sodium hydroxide. Other similar anionic surfactants are described in U.S. Pat. Nos. 2,486,921; 2,486,922; and 2,396,278, which are incorporated herein by reference in their entirety.

Exemplary anionic surfactants for use in the personal care composition include ammonium lauryl sulfate, ammonium laureth sulfate, ammonium C10-15 pareth sulfate, ammonium C10-15 alkyl sulfate, ammonium C11-15 alkyl sulfate, ammonium decyl sulfate, ammonium deceth sulfate, ammonium undecyl sulfate, ammonium undeceth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, sodium C10-15 pareth sulfate, sodium C10-15 alkyl sulfate, sodium C11-15 alkyl sulfate, sodium decyl sulfate, sodium deceth sulfate, sodium undecyl sulfate, sodium undeceth sulfate, potassium lauryl sulfate, potassium laureth sulfate, potassium C10-15 pareth sulfate, potassium C10-15 alkyl sulfate, potassium C11-15 alkyl sulfate, potassium decyl sulfate, potassium deceth sulfate, potassium undecyl sulfate, potassium undeceth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium cocoyl isethionate and combinations thereof. The anionic surfactant may be sodium lauryl sulfate or sodium laureth sulfate.

The composition of the present invention can also include anionic surfactants selected from the group consisting of:

a) R₁O(CH₂CHR₃O)_{y}SO₃M;

b) CH₃(CH₂)_{z}CHR₂CH₂O(CH₂CHR₃O)_{y}SO₃M; and

c) mixtures thereof,

where R₁ represents CH₃ (CH₂)₁₀, R₂ represents H or a hydrocarbon radical comprising 1 to 4 carbon atoms such that the sum of the carbon atoms in z and R₂ is 8, R₃ is H or CH₃, y is 0 to 7, the average value of y is about 1 when y is not zero (0), and M is a monovalent or divalent, positively-charged cation.

Suitable anionic alkyl sulfates and alkyl ether sulfate surfactants include, but are not limited to, those having branched alkyl chains which are synthesized from C8 to C18 branched alcohols which may be selected from the group consisting of: Guerbet alcohols, aldol condensation derived alcohols, oxo alcohols, F-T oxo alcohols and mixtures thereof. Non-limiting examples of the 2-alkyl branched alcohols include oxo alcohols such as 2-methyl-1-undecanol, 2-ethyl-1-decanol, 2-propyl-1-nonanol, 2-butyl 1-octanol, 2-methyl-1-dodecanol, 2-ethyl-1-undecanol, 2-propyl-1-decanol, 2-butyl-1-nonanol, 2-pentyl-1-octanol, 2-pentyl-1-heptanol, and those sold under the tradenames LIAL® (Sasol), ISALCHEM® (Sasol), and NEODOL® (Shell), and Guerbet and aldol condensation derived alcohols such as 2-ethyl-1-hexanol, 2-propyl-1-butanol, 2-butyl-1-octanol, 2-butyl-1-decanol, 2-pentyl-1-nonanol, 2-hexyl-1-octanol, 2-hexyl-1-decanol and those sold under the tradename ISOFOL® (Sasol) or sold as alcohol ethoxylates and alkoxylates under the tradenames LUTENSOL XP® (BASF) and LUTENSOL XL® (BASF).

The anionic alkyl sulfates and alkyl ether sulfates may also include those synthesized from C8 to C18 branched alcohols derived from butylene or propylene which are sold under the trade names EXXAL™ (Exxon) and Marlipal® (Sasol). This includes anionic surfactants of the subclass of sodium trideceth-n sulfates (STnS), where n is between about 0.5 and about 3.5. Exemplary surfactants of this subclass are sodium trideceth-2 sulfate and sodium trideceth-3 sulfate. The composition of the present invention can also include sodium tridecyl sulfate.

The composition of the present invention can also include anionic alkyl and alkyl ether sulfosuccinates and/or dialkyl and dialkyl ether sulfosuccinates and mixtures thereof. The dialkyl and dialkyl ether sulfosuccinates may be a C6-15 linear or branched dialkyl or dialkyl ether sulfosuccinate. The alkyl moieties may be symmetrical (i.e., the same alkyl moieties) or asymmetrical (i.e., different alkyl moieties). Nonlimiting examples include: disodium lauryl sulfosuccinate, disodium laureth sulfosuccinate, sodium bistridecyl sulfosuccinate, sodium dioctyl sulfosuccinate, sodium dihexyl sulfosuccinate, sodium dicyclohexyl sulfosuccinate, sodium diamyl sulfosuccinate, sodium diisobutyl sulfosuccinate, linear bis(tridecyl) sulfosuccinate and mixtures thereof.

The personal care composition may comprise a co-surfactant. The co-surfactant can be selected from the group consisting of amphoteric surfactant, zwitterionic surfactant, non-ionic surfactant and mixtures thereof. The co-surfactant can include, but is not limited to, lauramidopropyl betaine, cocoamidopropyl betaine, lauryl hydroxysultaine, sodium lauroamphoacetate, disodium cocoamphodiacetate, cocamide monoethanolamide and mixtures thereof.

The personal care composition may further comprise from about 0.25% to about 15%, from about 1% to about 14%, from about 2% to about 13% by weight of one or more amphoteric, zwitterionic, nonionic co-surfactants, or a mixture thereof.

Suitable amphoteric or zwitterionic surfactants for use in the personal care composition herein include those which are known for use in shampoo or other personal care cleansing. Non limiting examples of suitable zwitterionic or amphoteric surfactants are described in U.S. Pat. Nos. 5,104,646 and 5,106,609, which are incorporated herein by reference in their entirety.

Amphoteric co-surfactants suitable for use in the composition include those surfactants described as derivatives of aliphatic secondary and tertiary amines in which the aliphatic radical can be straight or branched chain and wherein one of the aliphatic substituents contains from about 8 to about 18 carbon atoms and one contains an anionic group such as carboxy, sulfonate, sulfate, phosphate, or phosphonate. Suitable amphoteric surfactant include, but are not limited to, those selected from the group consisting of: sodium cocaminopropionate, sodium cocaminodipropionate, sodium cocoamphoacetate, sodium cocoamphodiacetate, sodium cocoamphohydroxypropylsulfonate, sodium cocoamphopropionate, sodium cornamphopropionate, sodium lauraminopropionate, sodium lauroamphoacetate, sodium lauroamphodiacetate, sodium lauroamphohydroxypropylsulfonate, sodium lauroamphopropionate, sodium cornamphopropionate, sodium lauriminodipropionate, ammonium cocaminopropionate, ammonium cocaminodipropionate, ammonium cocoamphoacetate, ammonium cocoamphodiacetate, ammonium cocoamphohydroxypropylsulfonate, ammonium cocoamphopropionate, ammonium cornamphopropionate, ammonium lauraminopropionate, ammonium lauroamphoacetate, ammonium lauroamphodiacetate, ammonium lauroamphohydroxypropylsulfonate, ammonium lauroamphopropionate, ammonium cornamphopropionate, ammonium lauriminodipropionate, triethanolamine cocaminopropionate, triethanolamine cocaminodipropionate, triethanolamine cocoamphoacetate, triethanolamine cocoamphohydroxypropylsulfonate, triethanolamine cocoamphopropionate, triethanolamine cornamphopropionate, triethanolamine lauraminopropionate, triethanolamine lauroamphoacetate, triethanolamine lauroamphohydroxypropylsulfonate, triethanolamine lauroamphopropionate, triethanolamine cornamphopropionate, triethanolamine lauriminodipropionate, cocoamphodipropionic acid, disodium caproamphodiacetate, disodium caproamphoadipropionate, disodium capryloamphodiacetate, disodium capryloamphodipriopionate, disodium cocoamphocarboxyethylhydroxypropylsulfonate, disodium cocoamphodiacetate, disodium cocoamphodipropionate, disodium dicarboxyethylcocopropylenediamine, disodium laureth-5 carboxyamphodiacetate, disodium lauriminodipropionate, disodium lauroamphodiacetate, disodium lauroamphodipropionate, disodium oleoamphodipropionate, disodium PPG-2-isodecethyl-7 carboxyamphodiacetate, lauraminopropionic acid, lauroamphodipropionic acid, lauryl aminopropylglycine, lauryl diethylenediaminoglycine, and mixtures thereof

The composition may comprises a zwitterionic co-surfactant, wherein the zwitterionic surfactant is a derivative of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group such as carboxy, sulfonate, sulfate, phosphate or phosphonate. The zwitterionic surfactant can be selected from the group consisting of: cocamidoethyl betaine, cocamidopropylamine oxide, cocamidopropyl betaine, cocamidopropyl dimethylaminohydroxypropyl hydrolyzed collagen, cocamidopropyldimonium hydroxypropyl hydrolyzed collagen, cocamidopropyl hydroxysultaine, cocobetaineamido amphopropionate, coco-betaine, coco-hydroxysultaine, coco/oleamidopropyl betaine, coco-sultaine, lauramidopropyl betaine, lauryl betaine, lauryl hydroxysultaine, lauryl sultaine, and mixtures thereof.

Suitable nonionic surfactants for use in the present invention include those described in McCutcheion's Detergents and Emulsifiers, North American edition (1986), Allured Publishing Corp., and McCutcheion's Functional Materials, North American edition (1992). Suitable nonionic surfactants for use in the personal care compositions of the present invention include, but are not limited to, polyoxyethylenated alkyl phenols, polyoxyethylenated alcohols, polyoxyethylenated polyoxypropylene glycols, glyceryl esters of alkanoic acids, polyglyceryl esters of alkanoic acids, propylene glycol esters of alkanoic acids, sorbitol esters of alkanoic acids, polyoxyethylenated sorbitor esters of alkanoic acids, polyoxyethylene glycol esters of alkanoic acids, polyoxyethylenated alkanoic acids, alkanolamides, N-alkylpyrrolidones, alkyl glycosides, alkyl polyglucosides, alkylamine oxides, and polyoxyethylenated silicones.

The co-surfactant can be a non-ionic surfactant selected from the alkanolamides group including: Cocamide, Cocamide Methyl MEA, Cocamide DEA, Cocamide MEA, Cocamide MIPA, Lauramide DEA, Lauramide MEA, Lauramide MIPA, Myristamide DEA, Myristamide MEA, PEG-20 Cocamide MEA, PEG-2 Cocamide, PEG-3 Cocamide, PEG-4 Cocamide, PEG-5 Cocamide, PEG-6 Cocamide, PEG-7 Cocamide, PEG-3 Lauramide, PEG-5 Lauramide, PEG-3 Oleamide, PPG-2 Cocamide, PPG-2 Hydroxyethyl Cocamide, PPG-2 Hydroxyethyl Isostearamide and mixtures thereof.

Representative polyoxyethylenated alcohols include alkyl chains ranging in the C9-C16 range and having from about 1 to about 110 alkoxy groups including, but not limited to, laureth-3, laureth-23, ceteth-10, steareth-10, steareth-100, beheneth-10, and commercially available from Shell Chemicals, Houston, Tex. under the trade names Neodol® 91, Neodol® 23, Neodol® 25, Neodol® 45, Neodol® 135, Neodo® l 67, Neodol® PC 100, Neodol® PC 200, Neodol® PC 600, and mixtures thereof.

Also available commercially are the polyoxyethylene fatty ethers available commercially under the Brij® trade name from Uniqema, Wilmington, Del., including, but not limited to, Brij® 30, Brij® 35, Brij® 52, Brij® 56, Brij® 58, Brij® 72, Brij® 76, Brij® 78, Brij® 93, Brij® 97, Brij® 98, Brij® 721 and mixtures thereof.

Suitable alkyl glycosides and alkyl polyglucosides can be represented by the formula (S)n-O—R wherein S is a sugar moiety such as glucose, fructose, mannose, galactose, and the like; n is an integer of from about 1 to about 1000, and R is a C8-C30 alkyl group. Examples of long chain alcohols from which the alkyl group can be derived include decyl alcohol, lauryl alcohol, myristyl alcohol, cetyl alcohol, stearyl alcohol, oleyl alcohol, and the like. Examples of these surfactants include alkyl polyglucosides wherein S is a glucose moiety, R is a C8-20 alkyl group, and n is an integer of from about 1 to about 9. Commercially available examples of these surfactants include decyl polyglucoside and lauryl polyglucoside available under trade names APG® 325 CS, APG® 600 CS and APG® 625 CS) from Cognis, Ambler, Pa. Also useful herein are sucrose ester surfactants such as sucrose cocoate and sucrose laurate and alkyl polyglucosides available under trade names Triton™ BG-10 and Triton™ CG-110 from The Dow Chemical Company, Houston, Tex.

Other nonionic surfactants suitable for use in the present invention are glyceryl esters and polyglyceryl esters, including but not limited to, glyceryl monoesters, glyceryl monoesters of C12-22 saturated, unsaturated and branched chain fatty acids such as glyceryl oleate, glyceryl monostearate, glyceryl monopalmitate, glyceryl monobehenate, and mixtures thereof, and polyglyceryl esters of C12-22 saturated, unsaturated and branched chain fatty acids, such as polyglyceryl-4 isostearate, polyglyceryl-3 oleate, polyglyceryl-2-sesquioleate, triglyceryl diisostearate, diglyceryl monooleate, tetraglyceryl monooleate, and mixtures thereof.

Also useful herein as nonionic surfactants are sorbitan esters. Sorbitan esters of C12-22 saturated, unsaturated, and branched chain fatty acids are useful herein. These sorbitan esters usually comprise mixtures of mono-, di-, tri-, etc. esters. Representative examples of suitable sorbitan esters include sorbitan monolaurate (SPAN® 20), sorbitan monopalmitate (SPAN® 40), sorbitan monostearate (SPAN® 60), sorbitan tristearate (SPAN® 65), sorbitan monooleate (SPAN® 80), sorbitan trioleate (SPAN® 85), and sorbitan isostearate.

Also suitable for use herein are alkoxylated derivatives of sorbitan esters including, but not limited to, polyoxyethylene (20) sorbitan monolaurate (Tween® 20), polyoxyethylene (20) sorbitan monopalmitate (Tween® 40), polyoxyethylene (20) sorbitan monostearate (Tween® 60), polyoxyethylene (20) sorbitan monooleate (Tween® 80), polyoxyethylene (4) sorbitan monolaurate (Tween® 21), polyoxyethylene (4) sorbitan monostearate (Tween® 61), polyoxyethylene (5) sorbitan monooleate (Tween® 81), and mixtures thereof, all available from Uniqema.

Also suitable for use herein are alkylphenol ethoxylates including, but not limited to, nonylphenol ethoxylates (Tergitol™ NP-4, NP-6, NP-7, NP-8, NP-9, NP-10, NP-11, NP-12, NP-13, NP-15, NP-30, NP-40, NP-50, NP-55, NP-70 available from The Dow Chemical Company, Houston, Tex.) and octylphenol ethoxylates (Triton™ X-15, X-35, X-45, X-114, X-100, X-102, X-165, X-305, X-405, X-705 available from The Dow Chemical Company, Houston, Tex.).

Also suitable for use herein are tertiary alkylamine oxides including lauramine oxide and cocamine oxide.

Non limiting examples of other anionic, zwitterionic, amphoteric, and non-ionic additional surfactants suitable for use in the personal care composition are described in McCutcheon's, Emulsifiers and Detergents, 1989 Annual, published by M. C. Publishing Co., and U.S. Pat. Nos. 3,929,678, 2,658,072; 2,438,091; 2,528,378, which are incorporated herein by reference in their entirety.

Suitable surfactant combinations comprise an average weight % of alkyl branching of from about 0.5% to about 30%, alternatively from about 1% to about 25%, alternatively from about 2% to about 20%. The surfactant combination can have a cumulative average weight % of C8 to C12 alkyl chain lengths of from about 7.5% to about 25%, alternatively from about 10% to about 22.5%, alternatively from about 10% to about 20%. The surfactant combination can have an average C8-C12/C13-C18 alkyl chain ratio from about 3 to about 200, alternatively from about 25 to about 175.5, alternatively from about 50 to about 150, alternatively from about 75 to about 125.

Deposition Aids

The shampoo compositions of the present invention may further comprise a deposition aid, such as a cationic polymer or cationic deposition polymer. Cationic polymers useful herein are those having an average molecular weight of at least about 5,000, alternatively from about 10,000 to about 10 million, and alternatively from about 100,000 to about 2 million.

The cationic polymer may be, including but not limited to a cationic guar polymer, has a weight average Molecular weight of less than 2.2 million g/mol, or from about 150 thousand to about 2.2 million g/mol, or from about 200 thousand to about 2.2 million g/mol, or from about 300 thousand to about 1.2 million g/mol, or from about 750,000 thousand to about 1 million g/mol. The cationic guar polymer may have a charge density of from about 0.2 to about 2.2 meq/g, or from about 0.3 to about 2.0 meq/g, or from about 0.4 to about 1.8 meq/g; or from about 0.5 meq/g to about 1.8 meq/g.

The cationic guar polymer may have a weight average Molecular weight of less than about 1.5 million g/mol, and has a charge density of from about 0.1 meq/g to about 2.5 meq/g. The cationic guar polymer may have a weight average molecular weight of less than 900 thousand g/mol, or from about 150 thousand to about 800 thousand g/mol, or from about 200 thousand to about 700 thousand g/mol, or from about 300 thousand to about 700 thousand g/mol, or from about 400 thousand to about 600 thousand g/mol or from about 150 thousand to about 800 thousand g/mol, or from about 200 thousand to about 700 thousand g/mol, or from about 300 thousand to about 700 thousand g/mol, or from about 400 thousand to about 600 thousand g/mol. The cationic guar polymer may have a charge density of from about 0.2 to about 2.2 meq/g, or from about 0.3 to about 2.0 meq/g, or from about 0.4 to about 1.8 meq/g; or from about 0.5 meq/g to about 1.5 meq/g.

Suitable cationic polymers include, for example, copolymers of vinyl monomers having cationic amine or quaternary ammonium functionalities with water soluble spacer monomers such as acrylamide, methacrylamide, alkyl and dialkyl acrylamides, alkyl and dialkyl methacrylamides, alkyl acrylate, alkyl methacrylate, vinyl caprolactone, and vinyl pyrrolidone. Other suitable spacer monomers include vinyl esters, vinyl alcohol (made by hydrolysis of polyvinyl acetate), maleic anhydride, propylene glycol, and ethylene glycol. Other suitable cationic polymers useful herein include, for example, cationic celluloses, cationic starches, and cationic guar gums. A nonlimiting example of a cationic polymer is guar hydroxypropyltrimonium chloride.

The cationic polymer can be included in the hair care compositions of the present invention at a level of from about 0.001 wt. % to about 10 wt. %. In the present invention, the cationic polymer may be present in an amount up to about 5 wt % based on the weight of the composition.

### Aqueous Carrier

The personal care composition comprises an aqueous carrier. Accordingly, the formulations of the personal care composition can be in the form of pourable liquids (under ambient conditions). Such compositions will therefore typically comprise an aqueous carrier, which is present at a level of from about 20 wt. % to about 95 wt. %, or from about 60 wt. % to about 85 wt. %. The aqueous carrier may comprise water, or a miscible mixture of water and organic solvent, and in one aspect may comprise water with minimal or no significant concentrations of organic solvent, except as otherwise incidentally incorporated into the composition as minor ingredients of other components.

The aqueous carriers useful in the personal care composition include water and water solutions of lower alkyl alcohols and polyhydric alcohols. The lower alkyl alcohols useful herein are monohydric alcohols having 1 to 6 carbons, in one aspect, ethanol and isopropanol. The polyhydric alcohols useful herein include propylene glycol, dipropylene glycol, hexylene glycol, glycerin, and propane diol.

### Emulsifiers

In cases where the personal care composition does not include a gel matrix, the 1,2-diol can be pre-emulsified before it is added in the personal care composition. Emulsifiers selection for each conditioning active is guided by the Hydrophilic-Lipophilic-Balance value (HLB value) of emulsifiers. Suitable range of HLB value is 6-16, more preferably 8-14. Emulsifiers with an HLB higher than 10 are water soluble. Emulsifiers with low HLB are lipid soluble. To obtain suitable HLB value, a mixture of two or more emulsifiers may be used. Suitable emulsifiers include non-ionic, cationic, anionic and amphoteric emulsifiers.

Rheology Modifier/Thickener

The personal care compositions mentioned above may also contain one or more rheology modifier/thickener to adjust the rheological characteristics of the composition for better feel, in-use properties and the suspending stability of the composition. For example, the rheological properties are adjusted so that the composition remains uniform during its storage and transportation and it does not drip undesirably onto other areas of the body, clothing or home furnishings during its use. Any suitable rheology modifier can be used. Further, the leave-on treatment may comprise from about 0.01% to about 3% of a rheology modifier, alternatively from about 0.1% to about 1% of a rheology modifier,

The one or more rheology modifier may be selected from the group consisting of polyacrylamide thickeners, cationically modified polysaccharides, associative thickeners, and mixtures thereof. Associative thickeners include a variety of material classes such as, for example: hydrophobically modified cellulose derivatives; hydrophobically modified alkoxylated urethane polymers, such as PEG-150/decyl alcohol/SMDI copolymer, PEG-150/stearyl alcohol/SMDI copolymer, polyurethane-39; hydrophobically modified, alkali swellable emulsions, such as hydrophobically modified polypolyacrylates, hydrophobically modified polyacrylic acids, and hydrophobically modified polyacrylamides; hydrophobically modified polyethers. These materials may have a hydrophobe that can be selected from cetyl, stearyl, oleayl, and combinations thereof, and a hydrophilic portion of repeating ethylene oxide groups with repeat units from 10-300, alternatively from 30-200, and alternatively from 40-150. Examples of this class include PEG-120-methylglucose dioleate, PEG-(40 or 60) sorbitan tetraoleate, PEG-150 pentaerythrityl tetrastearate, PEG-55 propylene glycol oleate, PEG-150 distearate.

Non-limiting examples of additional rheology modifiers include acrylamide/ammonium acrylate copolymer (and)polyisobutene (and) polysorbate 20; acrylamide/sodium acryloyldimethyl taurate copolymer/isohexadecane/polysorbate 80; acrylates copolymer; acrylates/beheneth-25 methacrylate copolymer; acrylates/C10-C30 alkyl acrylate crosspolymer; acrylates/steareth-20 itaconate copolymer; ammonium polyacrylate/Isohexadecane/PEG-40 castor oil; C12-16 alkyl PEG-2 hydroxypropylhydroxyethyl ethylcellulose (HM-EHEC); carbomer; crosslinked polyvinylpyrrolidone (PVP); dibenzylidene sorbitol; hydroxyethyl ethylcellulose (EHEC); hydroxypropyl methylcellulose (HPMC); hydroxypropyl methylcellulose (HPMC); hydroxypropylcellulose (HPC); methylcellulose (MC); methylhydroxyethyl cellulose (MEHEC); PEG-150/decyl alcohol/SMDI copolymer; PEG-150/stearyl alcohol/SMDI copolymer; polyacrylamide/C13-14 isoparaffin/laureth-7; polyacrylate 13/polyisobutene/polysorbate 20; polyacrylate crosspolymer-6; polyamide-3; polyquaternium-37 (and) hydrogenated polydecene (and) trideceth-6; polyurethane-39; sodium acrylate/acryloyldimethyltaurate/dimethylacrylamide; crosspolymer (and) isohexadecane (and) polysorbate 60; sodium polyacrylate. Exemplary commercially-available rheology modifiers include ACULYN™ 28, Klucel M CS, Klucel H CS, Klucel G CS, SYLVACLEAR AF1900V, SYLVACLEAR PA1200V, Benecel E 10M, Benecel K35M, Optasense RMC70, ACULYN™33, ACULYN™46, ACULYN™22, ACULYN™44, Carbopol Ultrez 20, Carbopol Ultrez 21, Carbopol Ultrez 10, Carbopol 1342, Sepigel™ 305, Simulgel™600, Sepimax Zen, and/or combinations thereof.

A non exclusive list of suitable thickeners for use herein include xanthan, guar, hydroxypropyl guar, scleroglucan, methyl cellulose, ethyl cellulose (commercially available as Aquacote (Registered trademark), hydroxyethyl cellulose (Natrosol (Registered trademark), carboxymethyl cellulose, hydroxypropylmethyl cellulose, microcrystalline cellulose, hydroxybutylmethyl cellulose, hydroxypropyl cellulose (Klucel (Registered trademark), hydroxyethyl ethyl cellulose, cetyl hydroxyethyl cellulose (Natrosol (Registered trademark Plus 330), N-vinylpyrollidone (Povidone (Registered trademark), Acrylates/Ceteth-20 Itaconate Copolymer (Structure (Registered trademark 3001), hydroxypropyl starch phosphate (Structure (Registered trademark ZEA), polyethoxylated urethanes or polycarbamyl polyglycol ester (e.g. PEG-150/Decyl/SMDI copolymer=Aculyn (Registered trademark 44, PEG-150/Stearyl/SMDI copolymer=Aculyn 46 (Registered trademark), trihydroxystearin (Thixcin (Registered trademark) acrylates copolymer (e.g. Aculyn (Registered trademark 33) or hydrophobically modified acrylate copolymers (e.g. Acrylates/Steareth-20 Methacrylate Copolymer=Aculyn (Registered trademark 22), and fatty alcohols, such as cetyl and stearyl alcohol, and combinations thereof.

### Conditioner Composition

The personal care composition of the present invention can be a hair conditioner. The hair conditioner composition delivers consumer desired benefits such as wet feel, combability, color retention, protection against hair damage, damage repair, dry feel, anti-frizz benefits, etc. shampooing in addition to scalp anti-dandruff efficacy benefit.

The conditioner composition may comprise rinse off conditioners. In addition, it may comprise other optional ingredients such as silicone or organic conditioning agents, hair health actives, anti-dandruff actives, and other ingredients.

Hair conditioners are typically applied on hair after rinsing the shampoo composition from the hair. The conditioner composition described herein delivers consumer desired hair conditioning in addition to anti-dandruff benefits.

The conditioner composition described herein may also comprise a conditioner gel matrix comprising (1) one or more high melting point fatty compounds, (2) a cationic surfactant system, and (3) a second aqueous carrier. After applying to the hair a conditioner composition, the conditioner is rinsed from the hair using water.

A. Cationic Surfactant System

The conditioner gel matrix of the conditioner composition includes a cationic surfactant system. The cationic surfactant system can be one cationic surfactant or a mixture of two or more cationic surfactants. The cationic surfactant system can be selected from: mono-long alkyl quaternized ammonium salt; a combination of mono-long alkyl quaternized ammonium salt and di-long alkyl quaternized ammonium salt; mono-long alkyl amidoamine salt; a combination of mono-long alkyl amidoamine salt and di-long alkyl quaternized ammonium salt, a combination of mono-long alkyl amindoamine salt and mono-long alkyl quaternized ammonium salt.

The cationic surfactant system can be included in the composition at a level by weight of from about 0.1% to about 10%, from about 0.5% to about 8%, from about 0.8% to about 5%, and from about 1.0% to about 4%.

### Mono-Long Alkyl Quaternized Ammonium Salt

The monoalkyl quaternized ammonium salt cationic surfactants useful herein are those having one long alkyl chain which has about 22 carbon atoms and in may be a C22 alkyl group. The remaining groups attached to nitrogen are independently selected from an alkyl group of from 1 to about 4 carbon atoms or an alkoxy, polyoxyalkylene, alkylamido, hydroxyalkyl, aryl or alkylaryl group having up to about 4 carbon atoms.

Mono-long alkyl quaternized ammonium salts useful herein are those having the formula

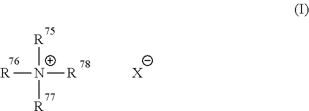

wherein one of R⁷⁵, R⁷⁶, R⁷⁷ and R⁷⁸ is selected from an alkyl group of 22 carbon atoms or an aromatic, alkoxy, polyoxyalkylene, alkylamido, hydroxyalkyl, aryl or alkylaryl group having up to about 30 carbon atoms; the remainder of R⁷⁵, R⁷⁶, R⁷⁷ and R⁷⁸ are independently selected from an alkyl group of from 1 to about 4 carbon atoms or an alkoxy, polyoxyalkylene, alkylamido, hydroxyalkyl, aryl or alkylaryl group having up to about 4 carbon atoms; and X⁻ is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether and/or ester linkages, and other groups such as amino groups. The longer chain alkyl groups, e.g., those of about 22 carbons, or higher, can be saturated or unsaturated. One of R⁷⁵, R⁷⁶, R⁷⁷ and R⁷⁸ can be selected from an alkyl group of about 22 carbon atoms, the remainder of R⁷⁵, R⁷⁶, R⁷⁷ and R⁷⁸ are independently selected from CH₃, C₂H₅, C₂H₄OH, and mixtures thereof; and X is selected from the group consisting of Cl, Br, CH₃OSO₃, C₂H₅OSO₃, and mixtures thereof.

Nonlimiting examples of such mono-long alkyl quaternized ammonium salt cationic surfactants include: behenyl trimethyl ammonium salt.

### Mono-Long Alkyl Amidoamine Salt

Mono-long alkyl amines are also suitable as cationic surfactants. Primary, secondary, and tertiary fatty amines are useful. Particularly useful are tertiary amido amines having an alkyl group of about 22 carbons. Exemplary tertiary amido amines include: behenamidopropyldimethylamine, behenamidopropyldiethylamine, behenamidoethyldiethylamine, behenamidoethyldimethylamin. Useful amines in the present invention are disclosed in U.S. Pat. No. 4,275,055, Nachtigal, et al. These amines can also be used in combination with acids such as l-glutamic acid, lactic acid, hydrochloric acid, malic acid, succinic acid, acetic acid, fumaric acid, tartaric acid, citric acid, l-glutamic hydrochloride, maleic acid, and mixtures thereof; and may be l-glutamic acid, lactic acid, and/or citric acid. The amines herein can be partially neutralized with any of the acids at a molar ratio of the amine to the acid of from about 1:0.3 to about 1:2, and/or from about 1:0.4 to about 1:1.

### Di-Long Alkyl Quaternized Ammonium Salt

Di-long alkyl quaternized ammonium salt can be combined with a mono-long alkyl quaternized ammonium salt or mono-long alkyl amidoamine salt. It is believed that such combination can provide easy-to rinse feel, compared to single use of a monoalkyl quaternized ammonium salt or mono-long alkyl amidoamine salt. In such combination with a mono-long alkyl quaternized ammonium salt or mono-long alkyl amidoamine salt, the di-long alkyl quaternized ammonium salts are used at a level such that the wt % of the dialkyl quaternized ammonium salt in the cationic surfactant system is in the range of from about 10% to about 50%, and/or from about 30% to about 45%.

The di-long alkyl quaternized ammonium salt cationic surfactants useful herein are those having two long alkyl chains having about 22 carbon atoms. The remaining groups attached to nitrogen are independently selected from an alkyl group of from 1 to about 4 carbon atoms or an alkoxy, polyoxyalkylene, alkylamido, hydroxyalkyl, aryl or alkylaryl group having up to about 4 carbon atoms.

Di-long alkyl quaternized ammonium salts useful herein are those having the formula (II):

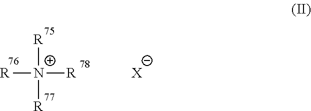

wherein two of R⁷⁵, R⁷⁶, R⁷⁷ and R⁷⁸ is selected from an alkyl group of from 22 carbon atoms or an aromatic, alkoxy, polyoxyalkylene, alkylamido, hydroxyalkyl, aryl or alkylaryl group having up to about 30 carbon atoms; the remainder of R⁷⁵, R⁷⁶, R⁷⁷ and R⁷⁸ are independently selected from an alkyl group of from 1 to about 4 carbon atoms or an alkoxy, polyoxyalkylene, alkylamido, hydroxyalkyl, aryl or alkylaryl group having up to about 4 carbon atoms; and X⁻ is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether and/or ester linkages, and other groups such as amino groups. The longer chain alkyl groups, e.g., those of about 22 carbons, or higher, can be saturated or unsaturated. One of R⁷⁵, R⁷⁶, R⁷⁷ and R⁷⁸ can be selected from an alkyl group of from 22 carbon atoms, the remainder of R⁷⁵, R⁷⁶, R⁷⁷ and R⁷⁸ are independently selected from CH₃, C₂H₅, C₂H₄OH, and mixtures thereof; and X is selected from the group consisting of Cl, Br, CH₃OSO₃, C₂H₅OSO₃, and mixtures thereof.

Such dialkyl quaternized ammonium salt cationic surfactants include, for example, dialkyl (C22) dimethyl ammonium chloride, ditallow alkyl dimethyl ammonium chloride, dihydrogenated tallow alkyl dimethyl ammonium chloride. Such dialkyl quaternized ammonium salt cationic surfactants also include, for example, asymmetric dialkyl quaternized ammonium salt cationic surfactants.

B. High Melting Point Fatty Compound

The conditioner gel matrix of the conditioner composition includes one or more high melting point fatty compounds. The high melting point fatty compounds useful herein may have a melting point of 25° C. or higher, and is selected from the group consisting of fatty alcohols, fatty acids, fatty alcohol derivatives, fatty acid derivatives, and mixtures thereof. It is understood by the artisan that the compounds disclosed in this section of the specification can in some instances fall into more than one classification, e.g., some fatty alcohol derivatives can also be classified as fatty acid derivatives. However, a given classification is not intended to be a limitation on that particular compound, but is done so for convenience of classification and nomenclature. Further, it is understood by the artisan that, depending on the number and position of double bonds, and length and position of the branches, certain compounds having certain carbon atoms may have a melting point of less than 25° C. Such compounds of low melting point are not intended to be included in this section. Nonlimiting examples of the high melting point compounds are found in International Cosmetic Ingredient Dictionary, Fifth Edition, 1993, and CTFA Cosmetic Ingredient Handbook, Second Edition, 1992.

Among a variety of high melting point fatty compounds, fatty alcohols are suitable for use in the conditioner composition. The fatty alcohols useful herein are those having from about 14 to about 30 carbon atoms, from about 16 to about 22 carbon atoms. These fatty alcohols are saturated and can be straight or branched chain alcohols. Suitable fatty alcohols include, for example, cetyl alcohol, stearyl alcohol, behenyl alcohol, and mixtures thereof.

High melting point fatty compounds of a single compound of high purity can be used. Single compounds of pure fatty alcohols selected from the group of pure cetyl alcohol, stearyl alcohol, and behenyl alcohol can also be used. By “pure” herein, what is meant is that the compound has a purity of at least about 90%, and/or at least about 95%. These single compounds of high purity provide good rinsability from the hair when the consumer rinses off the composition.

The high melting point fatty compound can be included in the conditioner composition at a level of from about 0.1% to about 20%, alternatively from about 1% to about 15%, and alternatively from about 1.5% to about 8% by weight of the composition, in view of providing improved conditioning benefits such as slippery feel during the application to wet hair, softness and moisturized feel on dry hair.

### Leave-on Treatment

The personal care composition of the present invention can be a leave-on treatment. The leave-on treatment composition delivers consumer desired hair conditioning or styling benefit in addition to scalp anti-dandruff efficacy benefit.

The leave-on treatment composition may comprise dry shampoos, mousses, pastes, gels, and milks. The leave-on treatment may also comprise (1) one or more rheology modifiers. In addition, it may comprise pother optional ingredients such as silicone or organic conditioning agents, thickeners, hair health actives, anti-dandruff actives, and other ingredients.

Accordingly, the formulations of the leave-on treatment can be in the form of pourable liquids (under ambient conditions).

In cases where the leave-on composition does not include a gel matrix, it is preferred that the composition is pre-emulsified before added in the personal care composition. In cases where the leave-on composition does not include a gel matrix, it is preferred that the composition also comprises a rheology modifier/thickener.

In the present invention, the leave-on treatment may involve the application of a 1% w/w solution of the materials in a mixture of water, emulsifier and a thickener (Sepigel 305). Preferred materials include 1,2-decanediol, 1,2-dodecanediol, 1,2-octanediol for 1-2-diols and silica silylate, salicylic acid, 2,4-dihydroxy benzoic acid, 4-chlororesorcinol, 1,2,4-Trihydroxybenzene and zinc carbonate for solid particles.

The azoxystrobin containing product may be a liquid, solid or powder or combinations thereof and can be dispensed from a container or can be a single use product. Non-limiting examples of single use products may include a discrete product that is in the form of a solid foam, capsule, pill, pod, sheet, film, tablet, compressed powder, encapsulated liquid, pouch or fibers. A powder may be dispensed from a container or delivered from an aerosol as a dry shampoo. The product may also be a liquid cleansing composition that is rinsed off including for cleansing skin or hair including shampoo, conditioners, body wash, or facial cleansing. The personal care product may be a deodorant in the form of a solid or an aerosol or pump spray.

### pH

The personal care compositions mentioned above may also comprise one or more pH adjusting material. The compositions may have a pH in the range from about 2 to about 10, at 25° C. The rinse-off conditioner composition, and/or leave-on treatment may have a pH in the range of from about 2 to about 6, alternatively from about 3.5 to about 5, alternatively from about 5.25 to about 7.

The personal care compositions mentioned above may further comprise one or more pH buffering agent. Suitable buffering agents are well known in the art and include for example ammonia/ammonium acetate mixture and monoethanolamine (MEA). The rinse-off conditioner composition may comprise citric acid, wherein the citric acid acts as a buffer.

### Optional Ingredients

The conditioner compositions, pre-wash compositions and/or leave-on treatments described herein may optionally comprise one or more additional components known for use in personal care or personal care products, provided that the additional components are physically and chemically compatible with the essential components described herein, or do not otherwise unduly impair product stability, aesthetics or performance. Such additional components are most typically those described in reference books such as the CTFA Cosmetic Ingredient Handbook, Second Edition, The Cosmetic, Toiletries, and Fragrance Association, Inc. 1988, 1992. Individual concentrations of such additional components may range from about 0.001 wt. % to about 10 wt. % by weight of the personal care compositions.

Non-limiting examples of additional components for use in the personal care compositions include conditioning agents, natural cationic deposition polymers, synthetic cationic deposition polymers, other anti-dandruff agents, particles, suspending agents, paraffinic hydrocarbons, propellants, viscosity modifiers, dyes, non-volatile solvents or diluents (water-soluble and water-insoluble), pearlescent aids, foam boosters, additional surfactants or nonionic cosurfactants, pediculocides, pH adjusting agents, perfumes, preservatives, proteins, skin active agents, sunscreens, UV absorbers, and vitamins.

1. Conditioning Agent

The personal care compositions may comprise one or more conditioning agents. Conditioning agents include materials that are used to give a particular conditioning benefit to hair. The conditioning agents useful in the personal care compositions of the present invention typically comprise a water-insoluble, water-dispersible, non-volatile, liquid that forms emulsified, liquid particles. Suitable conditioning agents for use in the personal care composition are those conditioning agents characterized generally as silicones, organic conditioning oils or combinations thereof, or those conditioning agents which otherwise form liquid, dispersed particles in the aqueous surfactant matrix.

One or more conditioning agents are present from about 0.01 wt. % to about 10 wt. %, from about 0.1 wt. % to about 8 wt. %, and from about 0.2 wt. % to about 4 wt. %, by weight of the composition.

### Silicone Conditioning Agent

The compositions of the present invention may contain one or more silicone conditioning agents. Examples of the silicones include dimethicones, dimethiconols, cyclic silicones, methylphenyl polysiloxane, and modified silicones with various functional groups such as amino groups, quaternary ammonium salt groups, aliphatic groups, alcohol groups, carboxylic acid groups, ether groups, epoxy groups, sugar or polysaccharide groups, fluorine-modified alkyl groups, alkoxy groups, or combinations of such groups. Such silicones may be soluble or insoluble in the aqueous (or non-aqueous) product carrier. In the case of insoluble liquid silicones, the polymer can be in an emulsified form with droplet size of about 10 nm to about 30 micrometers

### Organic Conditioning Materials

The conditioning agent of the compositions of the present invention may also comprise at least one organic conditioning material such as oil or wax, either alone or in combination with other conditioning agents, such as the silicones described above. The organic material can be nonpolymeric, oligomeric or polymeric. It may be in the form of oil or wax and may be added in the formulation neat or in a pre-emulsified form. Some non-limiting examples of organic conditioning materials include, but are not limited to: i) hydrocarbon oils; ii) polyolefins, iii) fatty esters, iv) fluorinated conditioning compounds, v) fatty alcohols, vi) alkyl glucosides and alkyl glucoside derivatives; vii) quaternary ammonium compounds; viii) polyethylene glycols and polypropylene glycols having a molecular weight of up to about 2,000,000 including those with CTFA names PEG-20 200, PEG-400, PEG-600, PEG-1000, PEG-2M, PEG-7M, PEG-14M, PEG-45M and mixtures thereof.

### Benefit Agents

The personal care composition may further comprise one or more additional benefit agents. The benefit agents comprise a material selected from the group consisting of anti-dandruff agents, anti-fungal agents, anti-itch agents, anti-bacterial agents, anti-microbial agents, moisturization agents, anti-oxidants, vitamins, lipid soluble vitamins, perfumes, brighteners, enzymes, sensates, attractants, dyes, pigments, bleaches, and mixtures thereof.

The personal care compositions of the present invention may be presented in typical personal care formulations. They may be in the form of solutions, dispersion, emulsions, powders, talcs, encapsulated, spheres, spongers, solid dosage forms, foams, and other delivery mechanisms. The compositions of the present invention may be hair tonics, leave-on hair products such as treatment, and styling products, rinse-off hair products such as hair conditioners, and treatment products; and any other form that may be applied to hair. The personal care composition may be a hair mask, cowash, hair wax, hair clay, hair food, hair milk, hair pudding and hair gels.

The personal care compositions may include compositions applied to hair on scalp, hair on other areas of the body such as face including beards, under arms, torso, legs, or other areas of skin with hair and may include beard washes and shave preparations.

The personal care compositions may be provided in the form of a porous, dissolvable solid structure, such as those disclosed in U.S. Patent Application Publication Nos. 2009/0232873; and 2010/0179083, which are incorporated herein by reference in their entirety. Accordingly, the personal care compositions comprise a chelant, a buffer system comprising an organic acid, from about 23% to about 75% surfactant; from about 10% to about 50% water soluble polymer; and optionally, from about 1% to about 15% plasticizer; such that the personal care composition is in the form of a flexible porous dissolvable solid structure, wherein said structure has a Percent open cell content of from about 80% to about 100%.

The personal care compositions may be in the form of a porous dissolvable solid structure comprising a chelant; a buffer system comprising an organic acid from about 23% to about 75% surfactant; wherein said surfactant has an average ethoxylate/alkyl ratio of from about 0.001 to about 0.45; from about 10% to about 50% water soluble polymer; and from about 1% to about 15% plasticizer; and wherein said article has a density of from about 0.03 g/cm³ to about 0.20 g/cm³.

The personal care compositions may be in the form of a viscous liquid comprising a chelant; a buffer system comprising an organic acid from 5-20% surfactant and a polycarboxylate rheology modifier; wherein the polycarboxylate is specifically chosen to be effective at the high electrolyte levels resulting from the incorporation of the key buffer system and chelant used for this invention. Non-limiting examples include acrylates/C10-C30 alkyl acrylate crosspolymers such as Carbopol EDT2020, 1342, 1382, etc. from Lubrizol. Rheology benefits of these actives may include stability, ease of dispensing, smoothness of spreading, etc.

The personal care compositions are generally prepared by conventional methods such as are known in the art of making the compositions. Such methods typically involve mixing of the ingredients in one or more steps to a relatively uniform state, with or without heating, cooling, application of vacuum, and the like. The compositions are prepared such as to optimize stability (physical stability, chemical stability, photostability) and/or delivery of the active materials. The personal care composition may be in a single phase or a single product, or the personal care composition may be in a separate phases or separate products. If two products are used, the products may be used together, at the same time or sequentially. Sequential use may occur in a short period of time, such as immediately after the use of one product, or it may occur over a period of hours or days.

### Methods

In Vivo Fungal Efficacy Testing

Subjects from all test groups will have Baseline scalp swabs for measurement of scalp Malassezia. Subjects will take home a test product(s) and will be instructed on use test products throughout the study. The test concludes after 2 or 3 azoxystrobin treatments with panelists' scalps being swabbed and samples collected. Malassezia is quantified from scalp surface swabs via qPCR. The change in Malassezia amount across time will be reported as % fungal reduction from baseline at the 2 or 3 treatment time point.

In Vivo Scalp Deposition Testing

Azoxystrobin deposition in-vivo on scalp can be determined by ethanol extraction of the agent after the scalp has been treated with an azoxystrobin containing composition either after application, after rinsing either immediately or at a delayed time point. The concentration of agent in the ethanol extraction solvent is measured by HPLC. Quantitation is made by reference to a standard curve. The concentration detected by HPLC is converted into an amount collected in grams by using the concentration multiplied by volume. The mass per volume concentration of the agent measured by HPLC is then converted to a mass per area amount deposited by multiplying the measured HPLC concentration by the volume of extraction solvent divided by the area of the scalp extracted.

In Vitro Antifungal Minimum Inhibitory Concentration (MIC) Testing

Malassezia furfur (CBS 7982) is maintained continuously as a culture at 31° C. in a 250-ml vent-capped polycarbonate Erlenmeyer flask by combining approximately 50 ml of mDixon growth medium and 2.5 ml of previously grown Malassezia culture. For each assay, Malassezia cells from 24-hour-old culture (approximately 7.5×10⁸ cells/ml) are diluted 500-fold into mDixon growth medium. Micropipettes are used to transfer 295 ul of diluted cells to each well of a Beckman 267007 polypropylene round-bottom deep-well plate.
Product forms are prepared for testing as concentrated stocks in water. Micropipettes are used to transfer 5 ul of appropriately diluted product form to the diluted Malassezia cells in the round-bottom deep-well plate. A semipermeable sealing film is applied to the plate which is then covered with water-soaked cotton batting. The deep-well plates are shaken at 31° C. on a Heidolph Titramax 1000 shaker at 1350 rpm for approximately 20 hours. The samples are mixed by micropipetting before transferring 200 ul of sample culture from each well to a Corning 3596 polystyrene plate. The plates are read immediately for absorbance at 600 nm using a Molecular Devices SpectraMax M5 plate reader. MIC values are presented as ppm of active.

### Results

This example demonstrates the ability of azoxystrobin to reduce Malassezia for scalp and skin benefits. Malassezia is the implicated fungus in dandruff development and other related skin diseases. It also interacts with the immune system of affected individuals to induce irritation, inflammation, and itch. The primary approach for treatment of dandruff, its associated symptoms such as flaking and itch and other such skin diseases involves application of antifungals for reduction of Malassezia on the scalp or skin.

| | | Statistical | In vivo scalp |
|---|---|---|---|
| | | Grouping for | Azoxystrobin |
| | % Fungal | % Fungal | deposition |
| Shampoo | Reduction** | Reduction | (ug/cm2)* |
| 0.25% Azoxystrobin | 89.4 | b | 0.077 |
| 0.5% Azoxystrobin | 97.0 | a | 0.270 |
| 0.5% Azoxystrobin (no | 94.1 | ab | 0.119 |
| cationic deposition | | | |
| polymer) | | | |
| 1.0% Azoxystrobin | 94.7 | ab | 0.513 |
| 1.0% Selenium sulfide (no | 96.0 | a | 0.150 |
| cationic deposition | | | |
| polymer) | | | |

*Panelists applied 10 ml of product and rinsed. Extractions are taken from the scalp at a 3 hour time point per method provided above.
**After 2 azoxystrobin treatments

The result of the qPCR analysis is analyzed with a Tobit regression model that included terms for treatment and baseline level. Test groups with different statistical grouping letters are statistically significant at p<0.05. 0.25% azoxystrobin shampoo reduced 89.4% of Malassezia on the scalp and 0.5% azoxystrobin shampoo reduced 97.0% Malassezia after 2 treatments compared to 96.0% reduction by the highly efficient industry standard and benchmark of 1.0% selenium sulfide in a commercially available shampoo. The results demonstrate that azoxystrobin formulated into a shampoo can deliver antifungal efficacy that is highly effective at reduction of scalp Malassezia. Azoxystrobin reduced the majority of Malassezia on the scalp after 2 treatments which may then forego the need for a 3^{rd} treatment. The in vivo deposition data demonstrates a high degree of potency at low levels of active amount delivered to the scalp surface. This antifungal efficacy exhibited by azoxystrobin indicates that it has comparable potency to marketed selenium sulfide shampoo, which is generally regarded as the most potent and efficacious active for anti-dandruff treatment.

The present invention may be directed to the use of azoxystrobin in a personal care composition for improving a dandruff condition. The present invention may be directed to the use of azoxystrobin in a personal care composition for reduction of dandruff. The present invention may be directed to the use of azoxystrobin as claimed in present claim set for reduction of dandruff.

The present invention demonstrates that at least 0.05% azoxystrobin provides a deposition of greater than about 0.01 ug/cm2 compared to at least 1% azoxystrobin which provides a deposition of greater than about 0.1 ug/cm2 wherein the antifungal efficacy of at least 0.5% azoxystrobin with less deposition per ug/cm2 is equal to the antifungal efficacy of at least 1% azoxystrobin with a greater deposition per ug/cm2.

The present invention may comprise azoxystrobin wherein at least 0.05% azoxystrobin provides a deposition of greater than about 0.01 ug/cm2. Further the present invention may demonstrate wherein the antifungal efficacy of at least 0.5% azoxystrobin with less deposition per ug/cm2 is equal to the antifungal efficacy of at least 1% azoxystrobin with a greater deposition per ug/cm2.

A dissolution and diffusion model is utilized to help dimension potent particle sizes for scalp delivered azoxystrobin particles. The model predicts where azoxystrobin concentration in sebum and when that concentration is above the minimal inhibitory concentration (MIC, with a value of about 0.2-0.9 ppm for azoxystrobin) for the fungus that causes dandruff (Malassezia). The model factors in azoxystrobin dissolution varying sebum thickness, sebum production rate and loss of soluble azoxystrobin into the scalp/stratum corneum over time. The table below shows two outputs of the model as particle size is varied: 1) the area under the concentration curve (azoxystrobin concentration in sebum divided by MIC integrated over time) and 2) the time in hours where the azoxystrobin concentration is sebum is above the MIC. It is observed from the simulations that as particle size increases starting at 1 um, the approximate number of hours and total area under the curve increases until the particle size of about 30-50 microns. Here a maximum in total area under the curve is achieved. Increasing particle size above about 70 um results in less total area under the curve and less maximal potency expected. The total residence time that azoxystrobin resides above the MIC continues to increase until about 190 um where both the total area under the curve and time in hours above the MIC drops to zero.

| | Total Area | Approximate Number |
|---|---|---|
| Mean | Under the Curve | of Hours that |
| Azoxystrobin | (Concentration of | Azoxystrobin Concentration |
| Particle Size | Azoxystrobin in sebum/ | is Above Minimum |
| (um) | MIC Integrated over time) | Inhibitory Concentration |
| 1 | 1512 | 2 |
| 10 | 14194 | 17 |
| 30 | 32378 | 76 |
| 50 | 31697 | 207 |
| 70 | 23646 | 452 |
| 90 | 16893 | 859 |
| 110 | 12406 | 1479 |
| 130 | 9475 | 2359 |
| 150 | 7495 | 3541 |
| 170 | 6092 | 5059 |
| 190 | 0 | 0 |

In the present invention the particle size of azoxystrobin may be less than or equal to about 5 microns; the particle size of azoxystrobin may be in the range of from about 0.5 microns to about 5 microns; the particle size of azoxystrobin may be in the range of from about 1 micron to about 3 microns. Alternatively, the particle size of azoxystrobin may be greater than about 5 microns; further the particle size of azoxystrobin may be less than about 100 microns; the particle size of azoxystrobin may be in the range of from about 10 microns to about 80 microns; the particle size of azoxystrobin may be in the range of from about 30 microns to about 50 microns. Alternatively, the particle size of azoxystrobin may be greater than or equal to about 100 microns; the particle size of azoxystrobin may be in the range of from about 100 microns to about 150 microns.

In the tables below, MIC data against Malassezia are provided for leave-on treatment examples and single unit dose examples of product forms. The dry shampoo aerosol spray exhibited an MIC of 1.56 ppm when tested in vitro against Malassezia. The dry shampoo aerosol foam exhibited an MIC of 0.781 ppm and the non-aerosol foam produced an MIC of 1.56 ppm. Single unit dose shampoo exhibited an MIC of 3.125 ppm and single unit dose conditioner produced an MIC of 1.56 ppm. These results demonstrate that azoxystrobin, when formulated into different product forms is highly effective against Malassezia. All product forms tested displayed MICs less than 5 ppm, which exemplifies the ability of azoxystrobin, formulated in a range of personal care product forms, to inhibit Malassezia growth in vitro with strong antifungal potency.

### EXAMPLES AND COMPOSITIONS

The following examples illustrate non-limiting examples of the invention described herein. The exemplified shampoos, rinse-off conditioners, leave on treatments, personal care cleansing, single unit dose compositions can be prepared by conventional formulation and mixing techniques. It will be appreciated that other modifications of the oxidative dyeing compositions and rinse-off conditioner compositions within the skill of those in the formulation art can be undertaken without departing from the spirit and scope of this invention. All parts, percentages, and ratios herein are by weight unless otherwise specified. Some components may come from suppliers as dilute solutions. The amount stated reflects the weight percent of the active material, unless otherwise specified.

The following examples further describe and demonstrate non-limiting examples within the scope of the present invention. The examples are given solely for the purpose of illustration and are not to be construed as limitations of the present invention, as many variations thereof are possible without departing from the spirit and scope of the invention. Where applicable, ingredients are identified by chemical or CTFA name, or otherwise defined below.

### Shampoo Examples

| | | Example | | | | | | |
|---|---|---|---|---|---|---|---|---|
| | Component | 1 | 2 | 3 | 4 | 5 | 6 | 7 |
| | Water | 81 | 81 | 85 | 84 | 78 | 78 | 77 |
| 1 | Sodium laureth-1 sulfate | 11 | 14 | 0 | 6 | 0 | 0 | 0 |
| 2 | Sodium laureth-3 sulfate | 0 | 0 | 0 | 0 | 8 | 8 | 8 |
| 3 | Sodium lauryl sulfate | 0 | 0 | 0 | 0 | 7 | 7 | 7 |
| 4 | Cocamidopropyl betaine | 1 | 0 | 0 | 7 | 2 | 2 | 2 |
| 5 | CMEA | 1 | 2 | 0 | 0 | 0 | 0 | 0 |
| 6 | Sodium lauroyl sarcosinate | 0 | 0 | 5 | 0 | 0 | 0 | 0 |
| 7 | Decyl glucoside | 0 | 0 | 5 | 0 | 0 | 0 | 0 |
| 8 | Sodium laureth sulfosuccinate | 0 | 0 | 3 | 0 | 0 | 0 | 0 |
| 9 | Hydroxypropyl methylcellulose | 0.3 | 0.1 | 0.2 | 0 | 0 | 0 | 0 |
| 10 | Guar Hydroxypropyltrimonium | 0 | 0 | 0 | 0 | 0.25 | 0.25 | 0.25 |
| | chloride | | | | | | | |
| 11 | Fragrance | 0.8 | 0.8 | 0.8 | 0.8 | 0.85 | 0.85 | 0.85 |
| 12 | Sodium benzoate | 0.25 | 0.25 | 0.25 | 0.25 | 0.25 | 0.25 | 0.25 |
| 13 | Citric acid | 0.3 | 0.4 | 0.2 | 0.3 | 0.3 | 0.3 | 0.3 |
| 14 | 1,10-Decanediol | 0 | 0 | 0.1 | 0 | 0 | 0 | 0 |
| | Azoxystrobin | 0.8 | 0.5 | 0.1 | 0.02 | 0.25* | 0.5* | 1* |
| 15 | Ethylene glycol distearate | 1.8 | 0 | 0 | 0 | 1.5 | 1.5 | 1.5 |
| 16 | Dimethiconol | 1 | 0 | 0 | 0 | 0.8 | 0.8 | 0.8 |
| 17 | Sodium chloride | 1 | 1 | 0 | 1 | 0.6 | 0.6 | 0.6 |

All above are on active basis; e.g. 11% SLE1S would require an addition of 44% of a 25% active SLE1S solution. The below table explains each Note from the above table

| | |
|---|---|
| 1 | Supplied at 26% active by Procter and Gamble |
| 2 | Supplied at 28% active by Procter & Gamble |
| 3 | Supplied at 29% active by Procter and Gamble |
| 4 | Supplied at 30% active by Evonik |
| 5 | Supplied at 85% active by BASF |
| 6 | Supplied at 30% active by Croda |
| 7 | Supplied as 50% active by BASF |
| 8 | Supplied as 35% active by Solvay |
| 9 | Supplied by Dow |
| 10 | Supplied by Ashland |
| 11 | Supplied by Procter and Gamble |
| 12 | Supplied by Emerald Kalama |
| 13 | Supplied by Archer Daniels Midland |
| 14 | Supplied by Symrise |
| 15 | Supplied by Evonik |
| 16 | Supplied by Wacker |
| 17 | Supplied by Morton Salt |
| * | Supplied by Jiangsu Agrochem |

### Rinse Off Conditioner Examples

| Component | Example | | | | | | | | |
|---|---|---|---|---|---|---|---|---|---|
| Raw Material Name | 8 | 9 | 10 | 11 | | 12 | | 13 | |
| Water Purified | 86.63 | 91.92 | 92.37 | 91.72 | 95.47 | 92.27 | 94.42 | 97.77 | 94.87 |
| Glycerin | | | | 2 | | | | | |
| Acrylates/C10-30 Alkyl | | | | | | 0.35 | 1 | | |
| Acrylate Crosspolymer *1 | | | | | | | | | |
| Carbomer *2 | | | | | | 0.1 | | | |
| Hydroxypropyl Guar *3 | | | 0.35 | | | | | | |
| Guar | 0.05 | | | | | | | | |
| Hydroxypropyltrimonium | | | | | | | | | |
| Chloride *4 | | | | | | | | | |
| Hydroxyethylcellulose *5 | | 0.3 | | | | | | | |
| Polyquaternium-11 *6 | | | | 1.5 | | | | | |
| Polyquaternium-39 *7 | | | | | 1.2 | 0.2 | 0.1 | | |
| Polyacrylaminde & C13- | | | | | | | | 0.4 | 1.5 |
| 14 Isoparafin & Laureth- | | | | | | | | | |
| 7 *8 | | | | | | | | | |
| PEG-200 | | | | | | 2 | 1 | | |
| PPG-200 | | | | | | 2 | 1 | | |
| PEG-40 Hydrogenated | | | | 0.4 | 0.5 | | | | |
| Castor Oil | | | | | | | | | |
| Polysorbate 20 | | | | 0.2 | | | | | |
| Polysorbate 60 | | 0.1 | | | 0.2 | | | | |
| Polysorbate 80 | | | | | | | | | |
| Behentrimonium | | | 1.1 | | | | | | |
| Chloride *9 | | | | | | | | | |
| Behentrimonium | | | | | | | | | |
| Methosulfate *10 | | | | | | | | | |
| Stearamidopropyl | 2 | 0.5 | | | | | | | |
| Dimethylamine | | | | | | | | | |
| Dicetyldimoniun | | 0.5 | 0.35 | | | | | | |
| Chloride/Propylene | | | | | | | | | |
| Glycol *11 | | | | | | | | | |
| Cetyl Alcohol | 2.5 | 0.9 | 0.9 | | | | | | |
| Stearyl Alcohol | 4.5 | 0.1 | 2.3 | | | | | | |
| Glyceryl Stearate | | | 0.5 | | | | | | |
| Azoxystrobin | 0.5 | 0.5 | 0.5 | 0.5 | 0.5 | 0.5 | 0.5 | 0.5 | 0.5 |
| Dimethicone *12 | | 4 | | | | 1 | 0.1 | 0.2 | |
| Bis-Aminopropyl | 2 | | 0.5 | | 1 | | | | |
| Dimethicone *13 | | | | | | | | | |
| Silicone Emulsion *14 | | | | 2.5 | | | | | 2 |
| Sodium Benzoate | 0.25 | 0.25 | 0.25 | 0.25 | 0.25 | 0.25 | 0.25 | 0.25 | 0.25 |
| Benzyl Alcohol | 0.4 | 0.4 | 0.4 | 0.4 | 0.4 | 0.4 | 0.4 | 0.4 | 0.4 |
| Disodium EDTA | 0.13 | 0.13 | 0.13 | 0.13 | 0.13 | 0.13 | 0.13 | 0.13 | 0.13 |
| Triethanolamine | | | | 0.1 | | 0.5 | 0.8 | | |
| L-glutamic acid | 0.64 | | | | | | | | |
| Citric Acid | 0.1 | 0.1 | 0.05 | | 0.05 | | | 0.05 | 0.05 |
| Fragrance | 0.3 | 0.3 | 0.3 | 0.3 | 0.3 | 0.3 | 0.3 | 0.3 | 0.3 |
| TOTALS: | 100 | 100 | 100 | 100 | 100 | 100 | 100 | 100 | 100 |

| Component | Example | |
|---|---|---|
| Raw Material Name | 14 | 15 |

| | | | | |
|---|---|---|---|---|
| Water Purified | 83.92 | 89.82 | 42.93 | 40.93 |
| Glycerin | | | 50 | 50 |
| Behentrimonium Methosulfate *10 | 2.8 | 2.2 | | |
| Stearamidopropyl Dimethylamine | | | 1.13 | 1.13 |
| Dicetyldimoniun Chloride/Propylene Glycol *11 | | 0.7 | | |
| Cetyl Alcohol | 1.9 | 1.2 | 1.17 | 1.17 |
| Stearyl Alcohol | 4.7 | 2.9 | 2 | 2 |
| Sodium Benzoate | 0.25 | 0.25 | | |
| Benzyl Alcohol | 0.4 | 0.4 | 0.4 | 0.4 |
| Phenoxyethanol | 0.4 | 0.4 | 0.4 | 0.4 |
| Ethylhexyl Glycerin | | | 0.1 | 0.1 |
| Disodium EDTA | 0.13 | 0.13 | | |
| L-glutamic acid | | | 0.37 | 0.37 |
| Azoxystrobin | 2 | 1 | 1 | 1 |
| Bis-Aminopropyl Dimethicone *13 | 3 | 0.5 | | 2 |
| Fragrance | 0.5 | 0.5 | 0.5 | 0.5 |
| TOTALS: | 100 | 100 | 100 | 100 |

*1 Pemulene TR-1 from Lubrizol
*2 Carbopol 981 from Lubrizol
*3 Jaguar HP-105 from Solvay
*4 Jaguar Excel from Solvay
*5 Natrosol Hydroxyethylcellulose
*6 Gafquat 755N from Ashland
*7 Merquat 3330 from Lubrizol
*8 Sepigel 305 from Seppic
*9 Supplied as 80% from Clariant
*10 Supplied as 80% from Croda
*11 Varisoft 432 PPG from Evonik
*12 XF49-703 from Momentive
*13 Y-14945 from Momentive
*14 Belsil ADM 6060 from Wacker

### Leave on Treatment Examples

### Soluble

| | Dry Shampoo |
|---|---|
| % active in finished | Aerosol Spray |
| product | Example 16 |
| Hydrocarbon Propellant | 55.00 |
| (Propane/Isobutane | |
| A-46 blend, supplied | |
| by Aeropres) | |
| Ethanol | 36.76 |
| Azoxystrobin | 0.10 |
| (supplied by | |
| Jiangsu Agrochem) | |
| Tapioca Starch | 6.01 |
| Polymethylsilsesquioxane | |
| (DryFlo TS supplied | |
| by Nouryon) | |
| Tapioca Starch | 1.76 |
| (Tapioca Pure supplied | |
| by Nouryon) | |
| Silica (Aerosil 200 | 0.25 |
| supplied by Evonik) | |
| Perfume | 0.12 |
| In vitro antifungal | 1.56 ppm |
| activity (MIC) | |
| | Non-Aerosol |
| % active in finished | Dry Shampoo |
| product | Spray |
| Water | 44.28 |
| Ethanol | 50.00 |
| Azoxystrobin | 0.10 |
| Tapioca Starch | 5.00 |
| Polymethylsilsesquioxane | |
| (DryFlo TS supplied | |
| by Nouryon) | |
| Silica (Aerosil 200 | 0.50 |
| supplied by Evonik) | |
| Perfume | 0.12 |

### May be Soluble

| | Non-Aerosol |
|---|---|
| % active in finished | Foam |
| product | Example 17 |
| Water | 78.60 |
| Propylene Glycol | 15.00 |
| Polyquatemium-11 | 0.75 |
| (Gafquat 755 N-O | |
| supplied by Ashland) | |
| Phenoxyethanol | 0.50 |
| Hexanediol and | 0.30 |
| Caprylyl Glycol | |
| (Symdiol 68 supplied | |
| by Symrise) | |
| PEG-40 Hydrogenated | 0.40 |
| Castor oil | |
| Cetrimonium Chloride | 0.20 |
| Panthenol | 0.70 |
| Azoxystrobin (supplied | 0.10 |
| by Jiangsu Agrochem) | |
| Perfume | 0.13 |
| EDTA | 0.13 |
| Decyl Glucoside | 2.5 |
| Citric Acid | 0.10 |
| PEG-10 Dimethicone | 0.60 |
| (Silsurf Di-1010 | |
| supplied by Siltech) | |
| In vitro antifungal | 1.56 ppm |
| activity (MIC) | |

### Suspended

| | Dry Shampoo |
|---|---|
| % active in finished | Foam |
| product | Example 18 |
| Water | 89.93 |
| Polyquaternium-68 | 0.50 |
| (Luviquat Supreme AT1 | |
| supplied by BASF) | |
| Polyquaternium-11 | 0.50 |
| (Gafquat 755 N-O | |
| supplied by Ashland) | |
| Acrylate | 0.50 |
| Methacrylamide | |
| Copolymer (Luviset One | |
| supllied by BASF) | |
| Phenoxyethanol | 0.47 |
| Tapioca Starch (Tapioca | 0.47 |
| Pure supplied by | |
| Nouryon) | |
| Hexanediol and Caprylyl | 0.30 |
| Glycol (Symdiol 68 | |
| supplied by Symrise) | |
| Aminomethyl Propanol | 0.19 |
| Perfume | 0.20 |
| Laureth-4 | 0.40 |
| Cetrimonium Chloride | 0.30 |
| Silica (Aerosil 200 | 0.14 |
| supplied by Evonik) | |
| Azoxystrobin (supplied | 0.10 |
| by Jiangsu Agrochem) | |
| Hydrocarbon propellant | 6.00 |
| (Isobutane/Propane | |
| blend A-70 supplied by | |
| Aeropres) | |
| In vitro antifungal | 0.781 ppm |
| activity (MIC) | |
| % active in finished | Volumizing |
| product | Mousse |
| Water | 89.49 |
| Polyquaternium-4 | 2.93 |
| (Celquat L-200 supplied | |
| by Nouryon) | |
| DMDM Hydantoin | 0.38 |
| C9-11 Pareth-8 | 0.24 |
| Disodium EDTA | 0.10 |
| Perfume | 0.27 |
| Propylene Glycol | 0.49 |
| Azoxystrobin | 0.10 |
| Hydrocarbon propellant | 6.00 |
| (Isobutane/Propane | |
| blend A-70 supplied by | |
| Aeropres) | |

| | Male Styling |
|---|---|
| % active in finished | Paste |
| product | Example 19 |
| Water | 59.60 |
| Glycerin | 7.70 |
| Ceresin (Ceresin Wax supplied by | 7.00 |
| TH C Tramm) | |
| Carnauba Wax | 3.00 |
| Octylacrylamide/Acrylates/ | 3.00 |
| Butylaminoethyl Methacrylate | |
| Copolymer (Amphomer supplied | |
| by Nouryon) | |
| Petrolatum | 4.00 |
| Dicaprylyl Maleate | 1.50 |
| Polyvinylpyrrolidone (Luviskol | 2.00 |
| supllied by BASF) | |
| Ethanol | 2.00 |
| Cetearyl Alcohol | 2.00 |
| Cetyl Alcohol | 2.00 |
| Ceteareth-25 | 2.00 |
| Mineral Oil | 1.50 |
| Carbomer (Carbomer 940 supplied | 0.60 |
| by Lubrizol) | |
| Aminomethylpropanol | 0.40 |
| Hexanediol and Caprylyl | 0.80 |
| Glycol (Symdiol 68 | |
| supplied by Symrise) | |
| Perfume | 0.30 |
| Benzyl Alcogol | 0.50 |
| Azoxystrobin | 0.10 |

| % active in finished | Volumizing |
|---|---|
| product | Gel |
| Water | 86.59 |
| Sorbitol | 6.00 |
| VA/Crotonates Copolymer (Resyn 28-2930 | 3.00 |
| supplied by Nouryon) | |
| Octylacrylamide/Acrylates/ | 1.66 |
| Butylaminoethyl Methacrylate Copolymer | |
| (Amphomer supplied by Nouryon) | |
| Aminomethylpropanol | 0.75 |
| PEG-40 Hydrogenated Castor Oil | 0.40 |
| Hexanediol and Caprylyl Glycol (Symdiol | 0.80 |
| 68 supplied by Symrise) | |
| Perfume | 0.20 |
| Benzyl Alcohol | 0.50 |
| Azoxystrobin | 0.10 |

| | Moisturizing |
|---|---|
| % active in finished | Scalp Milk |
| product | Example 20 |
| Water | 82.40 |
| Amodimethicone (Dow Corning 939 | 5.00 |
| supplied by Dow Corning) | |
| Niacinamide | 2.50 |
| Quaternium-80 (Abilquat 3272 | 1.50 |
| supplied by Evonik) | |
| Panthenol | 0.90 |
| Propylene Glycol | 3.00 |
| Hexanediol and Caprylyl Glycol | 0.80 |
| (Symdiol 68 supplied by Symrise) | |
| Perfume | 0.50 |
| Benzyl Alcohol | 0.50 |
| Cetrimonium Chloride | 0.20 |
| Behentrimonium Chloride | 0.20 |
| Azoxystrobin | 0.10 |
| Disodium EDTA | 0.10 |
| Polyacrylamide & C13-14 Isoparafin | 0.80 |
| & Laureth 7 (Sepigel 305 supplied | |
| by Seppic) | |

### Personal Care Cleansing Compositions

| Component | Example |
|---|---|
| I: Personal Care Composition | Example 21 |
| 1) Aqueous Cleansing Phase | |
| Sodium Trideceth Sulfate | 9.14% |
| (sulfated from Trideceth-2, Solvay) | |
| Cocamidopropyl Betaine | 2.73% |
| (from BASF) | |
| Trideceth-3 | 1.46% |
| (from BASF) | |
| Sodium Chloride | 4.22% |
| Guar Hydroxypropyltrimonium Chlroide | 0.47% |
| (N-Hance CG-17 from Aqualon) | |
| Xanthan Gum | 0.33% |
| (Keltrol 1000 from CP Kelco) | |
| Acrylates/C10-30 Alkylacrylate Cross Polymer | 0.03% |
| (Aqupec SER-300C from Sumitomo) | |
| Methyl chloro isothiazolinone and methyl | 0.033% |
| isothiazolinone (Kathon CG, Rohm & Haas) | |
| Azoxystrobin | 0.8% |
| EDTA (Dissolvine NA 2x) | 0.31% |
| Sodium Benzoate | 0.14% |
| Perfume | 1.2% |
| Citric Acid, titrate | pH = 5.7 ± 0.2 |
| Water and minors | Q.S. |
| 2) Lipid Phase | |
| Petrolatum | 9.80% |
| Glyceryl Oleate | 0.20% |

Example 21 can be prepared through conventional mixing methods. First, a polymer-TDA-3 premix can be prepared by adding AQUPEC® SER 300 polymer, xanthan gum, and PEG-90M into tricedeth-3 (Iconal TDA-3 ethoxylated tridecyl alcohol). In the main mixing vessel, water, sodium chloride, N-Hance CG-17 polymer, cocamidopropyl betaine can be added with continuous mixing. Then, AQUPEC®-polymer-TDA-3 premix and sodium trideceth sulfate can be added, and the pH can be adjusted to about 5.7 using citric acid. Then, Azoxystrobin, preservatives and perfume can be added to the vessel, which can be mixed until homogeneous. In a separate vessel, add Petrolatum and Glyceryl oleate. Heat the vessel to about 85 C. Then, cool down the vessel to about 60 C. Add the lipid phase to the main mixing vessel with mixing. Keep mixing until homogeneous.

| Component | Example |
|---|---|
| Cleansing Body Wash Composition | Example 22 |
| Sodium Laureth-1 Sulfate | 9.35% |
| (from Procter & Gamble Company) | |
| Cocamidopropyl Betaine | 1.65% |
| (from BASF) | |
| Azoxystrobin | 0.05% |
| Methyl chloro isothiazolinone and methyl | 0.033% |
| isothiazolinone (Kathon CG, Rohm & Haas) | |
| EDTA (Dissolvine NA 2x) | 0.1% |
| Sodium Benzoate | 0.45% |
| Perfume | 1.2% |
| Citric Acid, titrate | pH = 5.7 ± 0.3 |
| Add NaCl to Adjust Viscosity | 10,000 cps |
| (Brookfield method) | |
| Water and minors | Q.S. |

Examples 22 can be prepared through conventional mixing methods. In the mixing vessel, add the following ingredient water, sodium laureth-3 sulfate, cocamidopropyl betaine, sodium lauryl sulfate with continuous mixing. Then, add Azoxystrobin, sodium benzoate, EDTA. Then, pH can be adjusted to about 5.7 using citric acid. Then Kathon preservative and perfume can be added to the vessel. The viscosity can be adjusted with sodium chloride to about 10,000 cps (Brookfield viscosity method). Keep mixing until homogenous.

| Component | Example |
|---|---|
| Liquid Hand Wash Composition | Example 23 |
| Sodium Lameth-3 Sulfate | 6.5% |
| (from Procter & Gamble Company) | |
| Sodium Lauryl Sulfate | 2.5% |
| (from Procter & Gamble Company) | |
| Cocamidopropyl Betaine | 1% |
| (from BASF) | |
| Azoxystrobin | 0.05% |
| Methyl chloro isothiazolinone and methyl | 0.033% |
| isothiazolinone (Kathon CG, Rohm & Haas) | |
| EDTA (Dissolvine NA 2x) | 0.1% |
| Sodium Benzoate | 0.45% |
| Perfume | 0.4% |
| Citric Acid, titrate | pH = 4.5 ± 0.3 |
| Add NaCl to Adjust Viscosity | 5,000 cps |
| (Brookfield method) | |
| Water and minors | Q.S. |

Examples 23 can be prepared through conventional mixing methods. In the mixing vessel, add the following ingredient water, sodium laureth-3 sulfate, cocamidopropyl betaine, sodium lauryl sulfate with continuous mixing. Add Azoxystrobin, sodium benzoate, EDTA with mixing until the particles are fully dissolved. Then, pH can be adjusted to about 4.5 using citric acid. Then Kathon preservative and perfume can be added to the vessel. The viscosity can be adjusted with sodium chloride to about 5,000 cps (Brookfield viscosity method). Keep mixing until homogenous.

| Component | Example |
|---|---|
| Traditional Bar Soap Composition | Example 24 |
| Sodium Tallowate | 66.8% |
| Sodium Palm Kernelate | 16.7% |
| Water | 12.0% |
| Glycerin | 1.6% |
| Fragrance | 1.0% |
| Sodium Chloride | 0.6% |
| Azoxystrobin | 0.5% |
| Palm Kernel Acid | 0.5% |
| Titanium Dioxide | 0.25% |
| EDTA | 0.05% |

### Preparation Method:

Bar soap compositions of the present invention can be made via a number of different processes known in the art. Preferably, the present compositions are made via a milling process, resulting in milled bar soap compositions. A typical milling process of manufacturing a bar soap composition includes: (a) a step in which the soap is made through either a continuous process (ConSap or continuous saponification process) or a batch-making process (i.e. neutralization process for hydrolysis fatty acid noodle or kettle process), (b) a vacuum drying step in which the soap is made into soap noodles. The soap noodles in the above example will consisting of sodium tallowate, sodium palm kernelate, water, glycerin, sodium chloride, palm kernel acid, and EDTA (c) an amalgamating step in which the soap noodles made in the first two steps are combined with the azoxystrobin, titanium dioxide, and fragrance (d) a refining step via a combination of refining plodders or mills in which a homogeneous mixture is obtained, (e) a plodding step in which the soap mixture is extruded as soap logs and then cut into soap plugs, and (f) a stamping step in which the soap plugs are stamped to yield the finished bar soap composition.

| Component | Example |
|---|---|
| Synthetic Bar Soap Composition | Example 25 |
| Sodium Cocoyl Isethionate | 55.0% |
| Stearic Acid | 18.0% |
| Sodium Tallowate | 8.0% |
| Coconut Acid | 5.9% |
| Water | 4.0% |
| Sodium Isethionate | 3.5% |
| Sodium Palm Kernelate | 2.0% |
| Fragrance | 1.5% |
| Cocamidopropyl Betaine | 0.6% |
| Sodium Laureth-1 Sulfate | 0.6% |
| Sodium Chloride | 0.4% |
| Titanium Dioxide | 0.25% |
| Glycerin | 0.2% |
| Azoxystrobin | 0.05% |

### Preparation Method:

Soap noodles as described in the previous example are combined with sodium cocoyl isethionate and fatty acid flakes, other powders, and liquid ingredients in an amalgamator where all ingredients are mixed together. The amalgamation step is then followed by a refining step via a combination of refining plodders or mills in which a homogeneous mixture is obtained. A plodding step follows in which the soap mixture is extruded as soap logs and then cut into soap plugs. Finally, a stamping step occurs in which the soap plugs are stamped to yield the finished bar soap composition.

### Single Unit Dose Examples

### Single Unit Dose—Shampoo

| | Example | Example | Example | Example | Example | Example |
|---|---|---|---|---|---|---|
| Ingredient | 26 | 27 | 28 | 29 | 30 | 31 |
| Distilled Water | 1.68 | 0.88 | 2.2 | 3.64 | 3.1 | 2.79 |
| Sodium Benzoate, NF | 0.3 | 0.3 | 0.29 | 0.26 | 0.26 | 0.3 |
| Guar | 0.92 | 0.92 | 0.89 | 0.81 | 0.79 | 0.92 |
| hydroxypropyltrimonium | | | | | | |
| Chloride¹ | | | | | | |
| Polyquatenium 76 ² | 0.17 | 0.17 | 0.16 | 0.15 | 0.15 | 0.17 |
| Polyvinyl Alcohol³ | 9.5 | 9.5 | 9.22 | 8.36 | 8.17 | 9.5 |
| Polyvinyl Alcohol⁴ | 9.5 | 9.5 | 9.22 | 8.36 | 8.17 | 9.5 |
| Lauryl Hydroxysultaine ⁵ | 11.79 | 11.79 | 11.44 | 10.38 | 10.14 | 11.79 |
| Sodium Chloride | 2.08 | 2.08 | 2.02 | 1.83 | 1.79 | 2.08 |
| Sodium Laureth 1 | 21.6 | 21.6 | 20.95 | 19.01 | 18.58 | 21.6 |
| Sulfate | | | | | | |
| Sodium Laureth 3 | 2.92 | 2.92 | 2.83 | 2.57 | 2.51 | 2.92 |
| Sulfate | | | | | | |
| Sodium Undecyl Sulfate | 13.28 | 13.28 | 10.50 | 11.69 | 11.42 | 13.28 |
| Amodimethicone⁶ | 5.21 | 5.21 | 5.05 | 4.58 | 4.48 | 5.21 |
| Fragrance | 7.44 | 7.44 | 7.22 | 6.55 | 6.40 | 7.44 |
| Sodium bicarbonate | 5.84 | 5.84 | 5.66 | 5.14 | 5.02 | 0 |
| Citric Acid, Anhydous | 5.47 | 5.47 | 5.31 | 4.81 | 4.70 | 0 |
| (Global) | | | | | | |
| Sodium Alumino Silicate | 2 | 2 | 1.94 | 1.76 | 1.72 | .0.0 |
| Polyvinyl pyrrolidone ⁹ | 0.1 | 0.1 | 0.1 | 0.1 | 0.10 | 0 |
| HiCap Starch | 0.0 | 0.0 | 0.0 | 0.0 | 7.5 | 7.5 |
| Azoxystrobin | 0.2 | 1 | 5 | 10 | 5.00 | 5 |

¹Jaguar C500 supplied by Solvay
² Mirapol AM-T supplied by Solvay
³PVA420H supplied by Kuraray
⁴PVA403 supplied by Kuraray
⁵ Mackham LHS supplied by Solvay
⁶Y-14945 Amino Fluid supplied by Momentive
7. PVP K-15 supplied by Ashland

| | Example | Example | Example | Example | Example | Example | Example |
|---|---|---|---|---|---|---|---|
| Ingredient | 32 | 33 | 34 | 35 | 36 | 37 | 38 |
| Distilled Water | 2.38 | 1.58 | 3.24 | 4.27 | 1.48 | 2.99 | 2.99 |
| Sodium Benzoate, NF | 0.34 | 0.35 | 0.33 | 0.30 | 0.35 | 0.33 | 0.33 |
| Guar | 1.06 | 1.06 | 1.02 | 0.93 | 1.06 | 1.02 | 1.02 |
| hydroxypropyltrimonium | | | | | | | |
| Chloride¹ | | | | | | | |
| Polyquatenium 76 ² | 0.20 | 0.20 | 0.18 | 0.17 | 0.20 | 0.18 | 0.18 |
| Polyvinyl Alcohol³ | 10.93 | 10.93 | 10.60 | 9.61 | 10.93 | 10.60 | 10.60 |
| Polyvinyl Alcohol⁴ | 10.93 | 10.93 | 10.60 | 9.61 | 10.93 | 10.60 | 10.60 |
| Lauryl Hydroxysultaine ⁵ | 13.56 | 13.56 | 13.16 | 11.94 | 13.56 | 13.16 | 18 |
| Disodium cocoglutamate | | | | | | | 32 |
| Sodium Chloride | 2.39 | 2.39 | 2.32 | 2.10 | 2.39 | 2.32 | 8 |
| Sodium Laureth 1 | 24.84 | 24.84 | 24.09 | 21.86 | 24.84 | 24.09 | 0 |
| Sulfate | | | | | | | |
| Sodium Laureth 3 | 3.36 | 3.36 | 3.25 | 2.96 | 3.36 | 3.25 | 0 |
| Sulfate | | | | | | | |
| Sodium Undecyl Sulfate | 15.27 | 15.27 | 12.08 | 13.44 | 15.27 | 12.08 | 0 |
| Amodimethicone⁶ | 5.99 | 5.99 | 5.81 | 5.27 | 5.99 | 5.81 | 5.81 |
| Fragrance | 8.55 | 8.56 | 8.30 | 7.53 | 8.56 | 8.30 | 8.30 |
| Benzoic acid | 0.00 | 0.00 | 0.00 | 0.00 | 0.1 | 0.25 | 0.25 |
| Azoxystrobin | 0.2 | 1 | 5 | 10 | 1 | 5 | 5 |

¹Jaguar C500 supplied by Solvay
² Mirapol AM-T supplied by Solvay
³PVA420H supplied by Kuraray
⁴PVA403 supplied by Kuraray
⁵ Mackham LHS supplied by Solvay
⁶Y-14945 Amino Fluid supplied by Momentive

### Solid Unit Dose—Shampoo

| Ingredient | Ex 39 |
|---|---|
| DI Water | 2.97 |
| Polyvinyl Alcohol ¹ | 28.88 |
| Sodium Laureth-1-Sulfate (SLE1S) ² | 26.58 |
| Sodium Undecyl Sulfate (NaC11) ³ | 16.32 |
| Lauryl Hydroxysultaine (LHS) ⁴ | 14.53 |
| Salt from LHS surfactant | 2.57 |
| Sodium Laureth-3-Sulfate (SLE3S) | 3.61 |
| Guar hydroxypropyltrimonium | 1.13 |
| Chloride (Jaquar C500 Guar) ⁵ | |
| Polyquatenium 76 (AM Triquat) ⁶ | 0.21 |
| Citric Acid | 1.83 |
| Sodium Benzoate | 0.38 |
| Azoxystrobin ⁷ | 1.00 |
| In vitro antifungal activity (MIC) | 3.125 ppm |

¹ Polyvinyl alcohol supplied by Kuraray
² SLE1S supplied by KLK Oleo
³ NaC11 supplied by Evonik
⁴ Lauryl Hydroxysultaine supplied by Solvay
⁵ Jaguar C500 Guar supplied by Ashland
⁶ AM Triquat Supplied by Solvay
⁷ Azoxystrobin supplied by Jiangsu Agrochem

### Single Unit Dose—Conditioner

| Ingredient | Ex. 40 | Ex. 41 | Ex. 42 | Ex. 43 | Ex. 44 | Ex. 45 |
|---|---|---|---|---|---|---|
| Water | 1.44 | 0.64 | 0.71 | 1.96 | 1.00 | 1.23 |
| Behenyltrimonium | 19.62 | 19.62 | 18.09 | 15.00 | 16.85 | 12.41 |
| Methosulfate¹ | | | | | | |
| Cetyl Alcohol | 43.15 | 43.15 | 39.72 | 13.00 | 15.00 | 12.72 |
| Stearyl Alcohol | 17.67 | 17.67 | 19.00 | 34.45 | 37.09 | 29.14 |
| Lauroyl Methyl | 6.51 | 6.51 | 6.38 | 7.05 | 7.59 | 5.86 |
| Glucamide ² | | | | | | |
| Polyvinylpyrrolidone, | | | | | 7.50 | 12.00 |
| K120³ | | | | | | |
| HiCap Starch | | | | | 7.5 | 12.5 |
| Polyvinyl alcohol⁴ | | | | 11.25 | | 10.00 |
| Fragrance | | | | | 5.0 | 5 |
| Amodimethicone⁵ | 7.50 | 7.50 | 7.50 | 7.50 | | 4.00 |
| Polyvinylpyrrolidone, | | | | 1.66 | 1.60 | |
| K30⁶ | | | | | | |
| Azoxystrobin | 0.2 | 1 | 5 | 5 | 5.0 | 5 |

¹Behentrimonium Methosulfate - IPA supplied by Croda
² GlucoTain ® Clean supplied by Clariant
³PVP K120 supplied by Ashland
⁴PVA505 supplied by Kuraray
⁵Y-14945 Amino Fluid supplied by Momentive
⁶PVP K30 supplied by Ashland

### Single Unit Dose—Conditioner

| Ingredient | Ex 46 |
|---|---|
| Water | 2.378 |
| Behenyltrimonium Methosulfate¹ | 18.22 |
| Cetyl Alcohol | 16.39 |
| Stearyl Alcohol | 40.09 |
| Lauroyl Methyl Glucamide ² | 8.20 |
| Polyvinylpyrrolidone, K120³ | 3.64 |
| Polyvinyl alcohol⁴ | 9.49 |
| Azoxystrobin ⁵ | 1 |
| In vitro antifungal activity (MIC) | 1.56 ppm |

¹Behentrimonium Methosulfate - IPA supplied by Croda
² GlucoTain ® Cleansupplied by Clariant
³PVP KI20 supplied by Ashland
⁴PVA505 supplied by Kuraray
⁵ Azoxystrobin supplied by Jiangsu Agrochem

### Methods of Making the Compositions

The formulations of the present invention may be present in typical personal care compositions. They may be in the form of solutions, dispersion, emulsions, powders, talcs, encapsulated, spheres, spongers, solid dosage forms, foams, and other delivery mechanisms. The composition of the present invention may be hair tonics, leave-on hair products such as conditioners, treatment, and styling products, and any other form that may be applied to the hair.

### Grooming Examples

### Moisturizer/Balm: Composition and Making Instructions

| | Example | Example | Example | Example | Example | Example |
|---|---|---|---|---|---|---|
| | 47 | 48 | 49 | 50 | 51 | 52 |
| Phase A | | | | | | |
| Water | Qs | Qs | Qs | Qs | Qs | Qs |
| Glycerin | 8.0% | 6.0% | 6.0% | 6.0% | 4.0% | 5.0% |
| Sorbitol | | | | | 2.0% | 2.0% |
| Disodium EDTA | 0.10% | 0.10% | 0.10% | 0.10% | 0.10% | 0.10% |
| Phase B | | | | | | |
| Cetearyl Alcohol | | | | | | |
| Emulgade Pl 68/50 ¹ | 0.20% | 0.20% | 0.20% | 0.20% | 0.20% | 0.20% |
| Cetiol SN ² | 5.0% | 5.0% | 5.0% | 3.50% | 3.50% | 5.0% |
| Cetyl Alcohol | 0.89% | 0.89% | 0.89% | 0.89% | 0.89% | 0.89% |
| PEG-100 Stearate | 0.10% | 0.10% | 0.10% | | 0.10% | 0.10% |
| Polymethylsilsesquioxane | 1.0% | | 1.0% | 1.0% | 1.0% | |
| Sorbitan Stearate | 0.10% | 0.10% | 0.10% | 0.10% | 0.10% | 0.10% |
| Steareth-2 | 0.10% | 0.10% | | | | |
| Steareth-21 | 0.10% | 0.10% | | | | |
| Stearic acid | 0.10% | 0.10% | 0.10% | | 0.10% | 0.10% |
| Stearyl Alcohol | 0.61% | 0.61% | 0.61% | 0.61% | 0.61% | 0.61% |
| Phase C | | | | | | |
| Aluminum Starch | 4.0% | 4.0% | 4.0% | 4.0% | 3.0% | 3.0% |
| Octenylsuccinate | | | | | | |
| Aristoflex AVC ³ | 0.75% | | 0.75% | 0.75% | 0.75% | 0.75% |
| Cyclomethicone | | | | | | |
| Dow Corning 1503 ⁴ | 2.0% | 2.0% | 2.0% | 2.0% | 2.0% | 2.0% |
| FD&C Blue No. 1 (CI | 0.0002% | 0.0002% | 0.0002% | 0.0002% | 0.0002% | 0.0002% |
| 42090) | | | | | | |
| Mackstat DM ⁵ | 0.08% | 0.08% | 0.08% | 0.08% | 0.08% | 0.08% |
| Glydant Plus Liquid ⁶ | 0.32% | 0.32% | 0.32% | 0.32% | 0.32% | 0.32% |
| KTZ Interfine Gold ⁷ | 0.10% | 0.10% | 0.10% | 0.10% | 0.10% | 0.10% |
| KTZ Interfine Green ⁸ | 0.50% | 0.50% | 0.50% | 0.50% | 0.25% | 0.25% |
| Sepiplus 400 ⁹ | | 0.75% | | | | |
| Azoxystrobin | 0.50% | 1.0% | 0.50% | 0.50% | 0.45% | 0.50% |
| Phase D | | | | | | |
| Fragrance | 0.80% | 0.80% | 0.80% | 0.80% | 0.80% | 0.80% |
| Menthol | 0.05% | 0.09% | 0.09% | 0.09% | 0.05% | 0.09% |
| Menthyl Lactate | 0.15% | 0.20% | 0.20% | 0.20% | 0.25% | 0.20% |
| | Example | Example | Example | Example | Example | Example |
| | 53 | 54 | 55 | 56 | 57 | 58 |
| Phase A | | | | | | |
| Water | Qs | Qs | Qs | Qs | Qs | Qs |
| Glycerin | 4.0% | 4.0% | 2.0% | 2.0% | 3.0% | 2.0% |
| Sorbitol | | | 2.0% | 2.0% | 2.0% | 2.0% |
| Disodium EDTA | 0.10% | 0.10% | 0.05% | 0.05% | 0.05% | 0.05% |
| Phase B | | | | | | |
| Cetearyl Alcohol | | | 1.0% | 1.0% | 1.0% | 1.0% |
| Emulgade Pl 68/50 ¹ | 0.20% | 0.20% | | | | |
| Cetiol SN ² | 5.0% | 5.0% | 1.50% | 1.50% | 1.50% | 3.50% |
| Cetyl Alcohol | 0.89% | 0.89% | | | | |
| PEG-100 Stearate | 0.10% | | | | | |
| Polymethylsilsesquioxane | 1.0% | 1.0% | | | 1.0% | 1.0% |
| Sorbitan Stearate | 0.10% | 0.10% | | | | |
| Steareth-2 | | | 1.80% | 1.80% | 1.80% | 1.80% |
| Steareth-21 | | | 0.90% | 0.90% | 0.90% | 0.90% |
| Stearic acid | 0.10% | | | | | |
| Stearyl Alcohol | 0.61% | 0.61% | | | | |
| Phase C | | | | | | |
| Aluminum Starch | 3.0% | 3.0% | | | | |
| Octenylsuccinate | | | | | | |
| Aristoflex AVC ³ | 0.75% | | 0.75% | | | |
| Cyclomethicone | | | 2.0% | 2.0% | | |
| Dow Corning 1503 ⁴ | 2.0% | 2.0% | 0.50% | 0.50% | 0.50% | 0.50% |
| FD&C Blue No. 1 (CI | 0.0002% | 0.0002% | 0.0002% | 0.0002% | 0.0002% | 0.0002% |
| 42090) | | | | | | |
| Mackstat DM ⁵ | 0.08% | 0.08% | 0.08% | 0.08% | 0.08% | 0.08% |
| Glydant Plus Liquid ⁶ | 0.32% | 0.32% | 0.32% | 0.32% | 0.32% | 0.32% |
| KTZ Interfine Gold ⁷ | 0.10% | 0.10% | | | 0.10% | |
| KTZ Interfine Green ⁸ | 0.50% | 0.50% | | | 0.25% | |
| Sepiplus 400 ⁹ | | 0.75% | | 1.0% | 1.0% | 0.75% |
| Azoxystrobin | 0.50% | 0.50% | 0.50% | 0.50% | 1.0% | 1.0% |
| Phase D | | | | | | |
| Fragrance | 0.80% | 0.80% | 0.40% | 0.40% | 0.40% | 0.40% |
| Menthol | 0.09% | 0.05% | | | 0.05% | 0.05% |
| Menthyl Lactate | 0.20% | 0.15% | | | 0.15% | 0.15% |

¹ Cetearyl Glucoside (and) Cetearyl Alcohol from Cognis Corp, Cincinnati, OH
² Cetearyl Isononanoate from Cognis Corp, Cincinnati, OH
³ Ammonium Acryloyldimethyltaurate/VP Copolymer from Clariant International AG, Switzerland.
⁴ Dimethicone (and) Dimethiconol from Dow Corning, Midland, MI
⁵ DMDM Hydantoin (and) Water from Rhodia Inc, Cranbury. NJ
⁶ DMDM Hydantoin (and) Iodopropynyl Butylcarbamate (and) Water from Lonza Group Ltd, Switzerland
⁷ Mica (and) Titanium dioxide (and) Tin Oxide from Kobo Products, Plainfield, NJ
⁸ Mica (and) Titanium dioxide from Kobo Products, Plainfield, NJ
⁹ Polyacrylate-13 (and) Polyisobutene (and) Polysorbate 20 from Seppic Inc, Fairfield, NJ

### Making Instructions for Moisturizer/Balm

Phase A materials are combined and heated in a container. Phase B materials are combined and heated in a separate container. Phase B is added to Phase A under high shear. The mixture of Phases A and B is cooled and the contents of Phase C are added with mixing. Phase D materials are blended in a separate container and added to the mixture of Phases A, B, and C. The final mixture is stirred until well blended. Qs means quantity sufficient to reach 100%.

### Washing Compositions: Composition and Making Instructions

| | Example | Example | Example | Example | Example |
|---|---|---|---|---|---|
| Ingredient | 59 | 60 | 61 | 62 | 63 |
| Water | Qs | Qs | Qs | Qs | Qs |
| Polyquaternium-10 (JR-400) | 0.25% | 0.25% | 0.25% | 0.25% | 0.25% |
| Disodium EDTA | 0.10% | 0.10% | 0.10% | 0.10% | 0.10% |
| PEG-100 | 0.20% | 0.20% | 0.20% | 0.20% | 0.20% |
| Sorbitol ¹³ | 1.99% | 1.99% | 1.99% | 1.99% | 1.99% |
| Glycerin | 1.99% | 1.99% | 1.99% | 1.99% | 1.99% |
| Sodium Lauroamphoacetate ¹⁴ | 3.19% | 3.19% | 3.19% | 3.19% | 3.19% |
| Sodium Trideceth Sulfate ¹⁵ | 3.24% | 3.24% | 3.24% | 3.24% | 3.24% |
| Sodium Myristol Sarcosinate ¹⁶ | 1.49% | 1.49% | 1.49% | 1.49% | 1.49% |
| Lauric Acid | 0.15% | 0.15% | 0.15% | 0.15% | 0.15% |
| Citric Acid | 0.50% | 0.50% | 0.50% | 0.50% | 0.50% |
| PEG-200 Hydrogenated | 2.99% | 2.99% | 2.99% | 2.99% | 2.99% |
| Glyceryl Palmitate/ | | | | | |
| PEG-7 Glyceryl Cocoate¹⁷ | | | | | |
| DMDM Hydantoin + | 0.40% | 0.40% | 0.40% | 0.40% | 0.40% |
| Iodopropynyl | | | | | |
| Butalcarbamate | | | | | |
| Perfume | 0.50% | 0.50% | 0.50% | 0.50% | 0.50% |
| Azoxystrobin | 0.25% | 0.25% | 0.25% | 0.25% | 1.0% |
| Menthol | | 0.10% | 0.10% | 0.08% | 0.04% |

¹³ Sorbitol 70% Solution
¹⁴ Sodium Lauroamphoacetate 32% Solution
¹⁵ Sodium Trideceth Sulfate 65% Solution
¹⁶ Sodium Myristol Sarcosinate 30% Solution
¹⁷Antil 200 - (Evonik/Goldschmidt)

### Making Instructions for Washing Composition

Weigh out the water in a vessel sufficient to hold the entire batch. Insert an overhead mixer with impeller into the vessel and increase agitation to create a vortex. Sprinkle the polymer into the vortex, ensure well dissolved. Heat batch to about 60° C. to hydrate the polymer. Add EDTA, PEGs, Sorbitol, Glycerin, Sodium Lauroamphoacetate, and the surfactants while heating. After batch is at 60° C., add the lauric acid. Continue mixing at 60° C. for at least five minutes. Adjust to a pH from 5.9-6.5 with citric acid and/or water. Remove heat, allow to cool to 35° C. Once below 35° C., add the perfume, preservatives and other ingredients.

### Pre-Shave Preparations: Composition and Making Instructions

| | Example | Example | Example | Eample | Example |
|---|---|---|---|---|---|
| Ingredient | 64 | 65 | 66 | 67 | 68 |
| Water | Qs | Qs | Qs | Qs | Qs |
| Sepigel 305 (Polyacrylamide | 2.00% | 2.00% | 2.00% | 2.00% | 2.00% |
| & C13-14 Isoparaffin & | | | | | |
| Laureth-7) | | | | | |
| Polyox N12K (PEG-23M) | 0.50% | 0.50% | 0.50% | 0.50% | 0.50% |
| Natrosol 250 HHR (HEC) | 0.80% | 0.80% | 0.80% | 0.80% | 0.80% |
| Glycerin 99.7% Usp/Fcc | 5.00% | 5.00% | 5.00% | 5.00% | 5.00% |
| Brij 35 (Laureth-23) | 2.00% | 2.00% | 2.00% | 2.00% | 2.00% |
| Disodium EDTA | 0.10% | 0.10% | 0.10% | 0.10% | 0.10% |
| Perfume | 0.15% | 0.15% | 0.15% | 0.15% | 0.15% |
| Glydant Plus | 0.20% | 0.20% | 0.20% | 0.20% | 0.20% |
| Azoxystrobin | 0.25% | 0.25% | 0.25% | 0.25% | 1.0% |
| Menthol | | 0.05% | 0.05% | 0.04% | 0.02% |

### Making Instructions for Pre-Shave Preparations

Weigh out the water in a vessel sufficient to hold the entire batch. Insert an overhead mixer with impeller into the vessel and increase agitation to create a vortex. Pre-blend the thickener and polymer powders. Sprinkle the polymer blend into the vortex until incorporated. Begin heating batch to 70 C to hydrate the polymers. Once the batch is at 70 C, add the oil and mix until uniform and dispersed. Add the liquid dispersion polymer to the batch and mix until uniform and hydrated, increasing rpms to maintain good mixing. Add the surfactant and mix until uniform and dispersed. Begin cooling batch to below 45 C. Once below 45 C, add the perfume, preservatives and other temperature-sensitive additives. Cool to below 35 C and QS with water

### Post Foaming Shave Gels: Composition and Making Instructions

Making instructions can be found in US 2006/0257349, paragraph 21.

| | Example | Example | Example | Example | Example |
|---|---|---|---|---|---|
| Ingredient | 69 | 70 | 71 | 72 | 73 |
| Sorbitol 70% Solution | 0.97% | 0.97% | 0.97% | 0.97% | 0.97% |
| Glycerin | 0.49% | | 0.49% | | 0.49% |
| Water | QS | QS | QS | QS | QS |
| hydroxyethyl cellulose¹⁸ | 0.49% | 0.49% | 0.49% | 0.49% | 0.49% |
| PEG-90M¹⁹ | 0.06% | 0.06% | 0.06% | 0.06% | 0.06% |
| PEG-23M²⁰ | 0.05% | 0.05% | 0.05% | 0.05% | 0.05% |
| PTFE²¹ | 0.15% | 0.15% | 0.15% | 0.15% | 0.15% |
| Palmitic acid | 7.53% | 7.53% | 7.53% | 7.53% | 7.53% |
| Stearic Acid | 2.53% | 2.53% | 2.53% | 2.53% | 2.53% |
| Glyceryl Oleate | 1.94% | 1.94% | 1.94% | 1.94% | 1.94% |
| Triethanolamine (99%) | 5.88% | 5.88% | 5.88% | 5.88% | 5.88% |
| Lubrajel Oil²² | 0.49% | 0.97% | 0.49% | 0.97% | 0.49% |
| Azoxystrobin | 0.25% | 0.25% | 0.25% | 0.25% | 1.0% |
| Menthol | | 0.11% | | 0.11% | |
| Fragrance | 0.87% | 0.87% | 0.87% | 0.87% | 0.87% |
| Other (e.g. Vit E, Aloe, etc.) | 0.10% | 0.10% | 0.10% | 0.10% | 0.10% |
| Dye | 0.10% | 0.10% | 0.10% | 0.10% | 0.10% |
| Isopentane (and) Isobutane | 2.85% | 2.85% | 2.85% | 2.85% | 2.85% |

¹⁸Available as Natrosol 250 HHR from Hercules Inc., Wilmington, DE
¹⁹Available as Polyox WSR-301 from Amerchol Corp., Piscataway, NJ
²⁰Available as Polyox WSR N-12K from Amerchol Corp., Piscataway, NJ
²¹Available as Microslip 519 from Micro Powders Inc., Tarrytown, NY
²²Available from Guardian Laboratories, Hauppauge, NY

### Making Instructions for Post-Foaming Shave Gels

The above-described compositions are made in the following manner: The water soluble polymers (polyethylene oxide, hydroxyethylcellulose) are added to water and mixed until the polymers are completely dissolved (about 30 min.). The aqueous mixture is then heated and the glyceryl oleate, sorbitol and fatty acids are added at about 60° C. and well mixed while the heating continues. At 80-85° C. the triethanolamine is added and mixed for about 20 minutes to form the aqueous soap phase, After cooling the aqueous soap phase to room temperature, the remaining components (i.e., Lubrajel, glycerin, fragrance, colorant, botanicals) are added to the aqueous soap phase and mixed well to form the gel concentrate. (Water may be added if required to bring the batch weight to 100%, thereby compensating for any water loss due to evaporation.) The concentrate is then combined with the volatile post-foaming agent under pressure within the filling line and filled into bottom-gassed aerosol cans with shearing through the valve under nitrogen pressure.

All percentages and ratios used herein are by weight of the total composition and all measurements made are at 25° C., unless otherwise designated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods.

In the examples, all concentrations are listed as weight percent, unless otherwise specified and may exclude minor materials such as diluents, filler, and so forth. The listed formulations, therefore, comprise the listed components and any minor materials associated with such components. As is apparent to one of ordinary skill in the art, the selection of these minors will vary depending on the physical and chemical characteristics of the particular ingredients selected to make the personal care composition.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular descriptions of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal care composition comprising azoxystrobin wherein at least about 0.05% azoxystrobin provides a deposition of greater than about 0.01 ug/cm2.

2. A personal care composition according to claim 1 wherein the antifungal efficacy of at least about 0.5% azoxystrobin with less deposition per ug/cm2 is equal to the antifungal efficacy of at least about 1% azoxystrobin with a greater deposition per ug/cm2.

3. A personal care composition according to claim 1 wherein azoxystrobin is present from about 0.02% to about 10%.

4. A persona care composition according to claim 1 wherein azoxystrobin is present from about 0.05% to about 2%.

5. A personal care composition according to claim 1, wherein azoxystrobin is present from about 0.1% to about 1%.

6. A personal care composition according to claim 1 comprising azoxystrobin wherein at 0.25% azoxystrobin results in greater than about 85% fungal reduction.

7. A personal care composition according to claim 1 comprising azoxystrobin wherein at about 0.5% to about 10% azoxystrobin results in greater than about 95% fungal reduction.

8. A personal care composition according to claim 1 comprising azoxystrobin wherein at about 1% azoxystrobin results in a greater than about 95% fungal reduction.

9. A personal care composition according to claim 1 wherein azoxystrobin at 0.5% results in a greater fungal reduction than selenium sulfide at 1%.

10. A personal care composition according to claim 1 wherein azoxystrobin results in a minimum inhibitory concentration (MIC) of less than 5 ppm against Malassezia when formulated into compositions of different forms.

11. A personal care composition according to claim 1 wherein the particle size of azoxystrobin is less than or equal to about 5 microns.

12. A personal care composition according to claim 1 wherein the particle size of azoxystrobin is from about 0.5 microns to about 5 microns.

13. A personal care composition according to claim 1 wherein the particle size of azoxystrobin is from about 1 micron to about 3 microns.

14. A personal care composition according to claim 1 wherein the particle size of azoxystrobin is greater than about 5 microns.

15. A personal care composition according to claim 1 wherein the particle size of azoxystrobin is less than about 100 microns.

16. A personal care composition according to claim 1 wherein the particle size of azoxystrobin is from about 10 microns to about 80 microns.

17. A personal care composition according to claim 1 wherein the particle size of azoxystrobin is from about 30 microns to about 50 microns.

18. A personal care composition according to claim 1 wherein the particle size of azoxystrobin is greater than or equal to about 100 microns.

19. A personal care composition according to claim 1 wherein the particle size of azoxystrobin is from about 100 microns to about 150 microns.

20. A personal care composition according to claim 1 wherein the personal care composition is a shampoo.

21. A personal care composition according to claim 1 wherein the personal care composition is a rinse off conditioner.

22. A personal care composition according to claim 1 wherein the personal care composition is a leave on treatment.

23. A personal care composition according to claim 1 wherein the personal care composition is selected from the group consisting of a dry shampoo aerosol spray; non-aerosol dry shampoo spray; non-aerosol foam; dry shampoo foam, mousse; styling paste, gel or milk and mixtures thereof.

24. A personal care composition according to claim 1 wherein the personal care composition is a single unit dose.

25. A personal care composition according to claim 1 wherein the personal care composition is selected from the group consisting of a personal care cleansing composition with an aqueous cleansing phase and a liquid phase, a cleansing body wash; a liquid hand wash, or a bar soap and mixtures thereof.

26. A personal care composition according to claim 1 wherein the composition comprises a surfactant.

27. A personal care composition according to claim 1 wherein the composition comprises an anionic, amphoteric, nonionic or zwitterionic surfactant or mixtures thereof.

28. A personal care composition according to claim 1 wherein the composition further comprises a polymer.

29. A personal care composition according to claim 28 wherein the polymer is a cationic polymer.

30. A personal care composition according to claim 1 wherein the composition further comprises a conditioning agent.

31. A personal care composition according to claim 30 wherein the conditioning agent is a silicone.

32. A personal care composition according to claim 1 further comprising from about 0.5% to about 7% of a perfume.
